(19) United States
(12) Patent Application Publication
(10) Pub. No.: US 2020/0078360 A1
(43) Pub. Date: Mar. 12, 2020

(54) SMALL MOLECULE DUAL INHIBITORS OF EGFR/PI3K AND USES THEREOF

(71) Applicant: The Regents of the University of Michigan, Ann Arbor, MI (US)

(72) Inventors: Christopher Emil WHITEHEAD, Ypsilanti, MI (US); Elizabeth ZIEMKE, Ann Arbor, MI (US); Judith SEBOLT-LEOPOLD, Ann Arbor, MI (US)

(21) Appl. No.: 16/347,496

(22) PCT Filed: Nov. 3, 2017
(86) PCT No.: PCT/US2017/059958

§ 371 Date: May 3, 2019

Related U.S. Application Data
(60) Provisional application No. 62/417,070, filed on Nov. 3, 2016.

(51) Int. Cl. A61K 31/517; A61K 31/437; A61K 31/519; A61K 31/506; A61K 31/4184; A61P 35/00

(52) U.S. Cl. CPC A61K 31/517; A61K 31/437; A61P 35/00; A61K 31/506; A61K 31/4184; A61K 31/519

(57) ABSTRACT

This invention is in the field of medicinal chemistry. In particular, the invention relates to anew class of small-molecules having a quinazoline structure which function as dual inhibitors EGFR and PI3K, and their use as therapeutics for the treatment of cancer (e.g., cancers associated with mutated KRAS and BRAF) (e.g., in combination with MAPK pathway inhibitors (e.g., BRAF inhibitors, MEK inhibitors, ERK inhibitors).

### CROSS-REFERENCE TO RELATED APPLICATIONS

The present application claims priority to U.S. Provisional Patent Application No. 62/417,070, filed Nov. 3, 2016, hereby incorporated by reference in its entirety.

### STATEMENT REGARDING FEDERALLY SPONSORED RESEARCH OR DEVELOPMENT

This invention was made with government support under grant CA155198 awarded by the National Institutes of Health. The government has certain rights in the invention.

### FIELD OF THE INVENTION

This invention is in the field of medicinal chemistry. In particular, the invention relates to a new class of small-molecules having a quinazoline structure which function as dual inhibitors EGFR and PI3K, and their use as therapeutics for the treatment of cancer (e.g., cancers associated with mutated KRAS and BRAF) (e.g., in combination with MAPK pathway inhibitors (e.g., BRAF inhibitors, MEK inhibitors, ERK inhibitors).

### INTRODUCTION

Cancers associated with KRAS and BRAF mutations are refractory to current treatment strategies. Indeed, patients diagnosed with cancers associated with KRAS and BRAF mutations (e.g., colorectal cancer, pancreatic cancer) have limited treatment options and poor prognosis.

Accordingly, improved methods for treating cancers associated with both KRAS and BRAF mutations are needed.

### SUMMARY OF THE INVENTION

The present invention addresses the need for improved methods for treating cancers associated with both KRAS and BRAF mutations. Indeed, experiments conducted during the course of developing embodiments for the present invention designed a new class of potent small-molecules having a quinazoline structure which function as dual inhibitors EGFR and PI3K. Such experiments further determined that a combination of such dual EGFR/PI3K inhibitors with MAPK pathway inhibitors (e.g., trametinib) resulted in a synergistic effect for treating cancers associated with both KRAS and BRAF mutations.

As such, the present invention provides a new class of small-molecules having a quinazoline structure which function as dual inhibitors EGFR and PI3K, and their use as therapeutics for the treatment of cancer and other diseases (e.g., in combination with MAPK pathway inhibitors).

Accordingly, the present invention contemplates that exposure of animals (e.g., humans) suffering from cancer (e.g., cancer associated with KRAS and BRAF mutations) (e.g., and/or cancer related disorders) to therapeutically effective amounts of drug(s) having a quinazoline structure (e.g., small molecules having a quinazoline structure) that inhibit the activity of both EGFR and PI3K will inhibit the growth of such cancer cells or supporting cells outright and/or render such cells as a population more susceptible to the cell death-inducing activity of cancer therapeutic drugs or radiation therapies.

Moreover, the present invention contemplates that such a therapeutic effect is enhanced (e.g., synergized) through combination treatment (e.g., simultaneous, non-simultaneous) with MAPK pathway inhibitors. Indeed, the present invention contemplates that dual inhibitors of EGFR and PI3K activity satisfy an unmet need for the treatment of multiple cancer types, either when administered as monotherapy to induce cell growth inhibition, apoptosis and/or cell cycle arrest in cancer cells, or when administered in a temporal relationship with additional agent(s), such as other cell death-inducing or cell cycle disrupting cancer therapeutic drugs (e.g., MAPK pathway inhibitors) or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of a compound of the present invention and a course of an anticancer agent (e.g., MAPK pathway inhibitor) produces a greater tumor response and clinical benefit in such animals compared to those treated with the compound or anticancer drugs/radiation alone. Since the doses for all approved anticancer drugs and radiation treatments are known, the present invention contemplates the various combinations of them with the present compounds.

The Applicants have found that certain quinazoline compounds function as dual inhibitors of EGFR and PI3K, and serve as therapeutics for the treatment of cancer and other diseases. Thus, the present invention relates to quinazoline compounds useful for inhibiting both EGFR and PI3K activity (e.g., thereby facilitating cell apoptosis), and increasing the sensitivity of cells to inducers of apoptosis and/or cell cycle arrest—administered alone or in combination with MAPK pathway inhibitors. Certain quinazoline compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, quinazoline compounds encompassed within Formula I are provided:

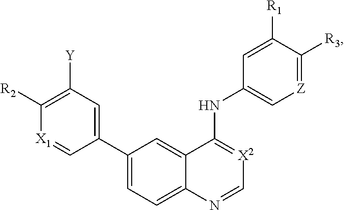

including pharmaceutically acceptable salts, solvates, and/or prodrugs thereof.

Formula I is not limited to a particular chemical moiety for R1, R2, R3, X1, X2, Y or Z. In some embodiments, the particular chemical moiety for R1, R2, R3, X1, X2, Y or Z independently include any chemical moiety that permits the resulting compound to inhibit both EGFR and PI3K activity. In some embodiments, the particular chemical moiety for R1, R2, R3, X, Y or Z independently include any chemical moiety that permits the resulting compound to effectively treat cancers associated with KRAS and BRAF mutations when administered alone or in combination with MAPK pathway inhibitors.

In some embodiments, R1 is hydrogen, halogen (e.g., Chlorine, Fluorine), or

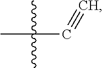 [SMILES: *C#C]

In some embodiments, R2 is hydrogen, halogen (e.g., Chlorine, Fluorine), or methoxy

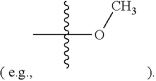 [SMILES: *OC]

In some embodiments, R3 is hydrogen, halogen (e.g., Chlorine, Fluorine), methoxy or pyridin-2-ylmethoxy (e.g.,

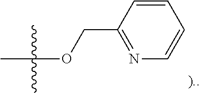 [SMILES: *OCc1ccccn1]

In some embodiments, X1 is Nitrogen or CH(e.g.,

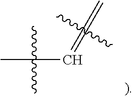 [SMILES: *C=*]

In some embodiments, X2 is Nitrogen or C-Nitrile(e.g.,

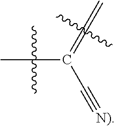

In some embodiments, Y is hydrogen, NHSO₂CH₃, or NHSO₂CH₂CH₂N(CH₃)₂ (e.g.,

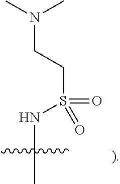 [SMILES: *NS(=O)(=O)CCN(C)C]

In some embodiments, Z is Nitrogen or CH (e.g.,

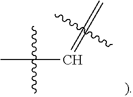 [SMILES: *C=*]

In some embodiments, the following compounds are contemplated for Formula I:

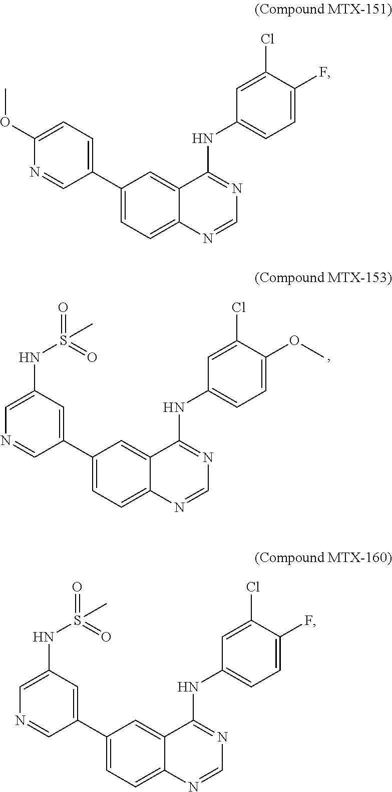 [SMILES: COc1ccc(-c2ccc3ncnc(Nc4ccc(F)c(Cl)c4)c3c2)cn1 ; COc1ccc(Nc2ncnc3ccc(-c4cncc(NS(C)(=O)=O)c4)cc23)cc1Cl ; CS(=O)(=O)Nc1cncc(-c2ccc3ncnc(Nc4ccc(F)c(Cl)c4)c3c2)c1]

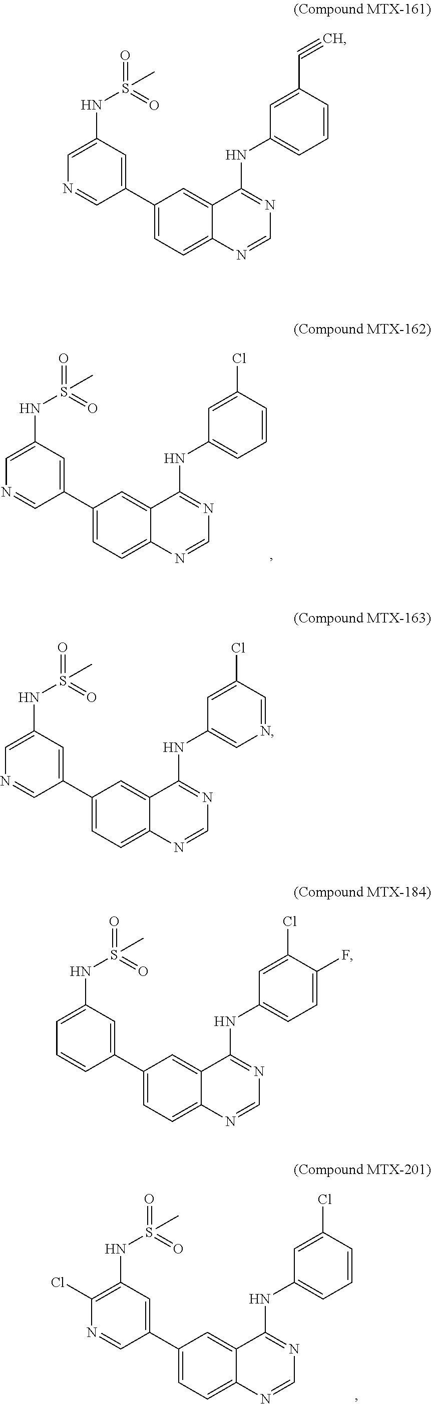 [SMILES: C#Cc1cccc(Nc2ncnc3ccc(-c4cncc(NS(C)(=O)=O)c4)cc23)c1 ; CS(=O)(=O)Nc1cncc(-c2ccc3ncnc(Nc4cccc(Cl)c4)c3c2)c1 ; CS(=O)(=O)Nc1cncc(-c2ccc3ncnc(Nc4cncc(Cl)c4)c3c2)c1 ; CS(=O)(=O)Nc1cccc(-c2ccc3ncnc(Nc4ccc(F)c(Cl)c4)c3c2)c1 ; CS(=O)(=O)Nc1cc(-c2ccc3ncnc(Nc4cccc(Cl)c4)c3c2)cnc1Cl]

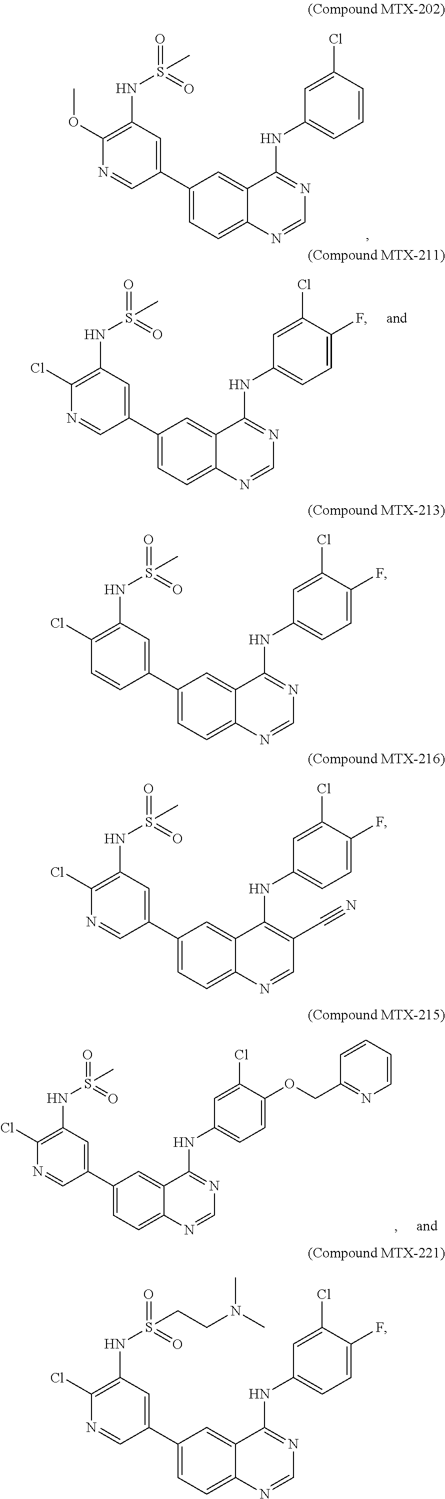 [SMILES: COc1ncc(-c2ccc3ncnc(Nc4cccc(Cl)c4)c3c2)cc1NS(C)(=O)=O ; CS(=O)(=O)Nc1cc(-c2ccc3ncnc(Nc4ccc(F)c(Cl)c4)c3c2)cnc1Cl ; CS(=O)(=O)Nc1cc(-c2ccc3ncnc(Nc4ccc(F)c(Cl)c4)c3c2)ccc1Cl ; CS(=O)(=O)Nc1cc(-c2ccc3ncc(C#N)c(Nc4ccc(F)c(Cl)c4)c3c2)cnc1Cl ; CS(=O)(=O)Nc1cc(-c2ccc3ncnc(Nc4ccc(OCc5ccccn5)c(Cl)c4)c3c2)cnc1Cl ; CN(C)CCS(=O)(=O)Nc1cc(-c2ccc3ncnc(Nc4ccc(F)c(Cl)c4)c3c2)cnc1Cl]

or a pharmaceutically acceptable salt, solvate, or prodrug thereof.

Table 1 (see, Examples) show IC50 values for specific compounds of the present invention for inhibiting EGFR and PI3K.

The invention further provides processes for preparing any of the compounds of the present invention through following skills well known in the art.

The invention also provides the use of compounds to induce cell cycle arrest and/or apoptosis in cells containing mutated forms of KRAS and BRAF (e.g., colorectal cancer, pancreatic, melanoma, non-small cell lung cancer, etc). The invention also relates to the use of compounds for sensitizing cells to additional agent(s), such as inducers of apoptosis and/or cell cycle arrest, and chemoprotection of normal cells through the induction of cell cycle arrest prior to treatment with chemotherapeutic agents.

The compounds of the invention are useful for the treatment, amelioration, or prevention of disorders, such as those responsive to induction of apoptotic cell death, e.g., disorders characterized by dysregulation of apoptosis, including hyperproliferative diseases such as cancer. In certain embodiments, the compounds can be used to treat, ameliorate, or prevent cancer that is characterized by resistance to cancer therapies (e.g., those cancer cells which are chemoresistant, radiation resistant, hormone resistant, and the like) (e.g., cancers associated with mutated BRAF and KRAS activity). In certain embodiments, the cancer is any type or form of cancer associated with aberrant BRAF and KRAS activity. In certain embodiments, the cancer is selected from, for example, colorectal cancer, pancreatic, melanoma, and non-small cell lung cancer.

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., anticancer agents or apoptosis-modulating agents, e.g., MAPK pathway inhibitors.

As described in the Examples, efforts conducted during the course of developing embodiments for the present invention resulted in the formation of novel small-molecules having a quinazoline structure that function as dual inhibitors of EGFR and PI3K. For example, Compound MTX-211

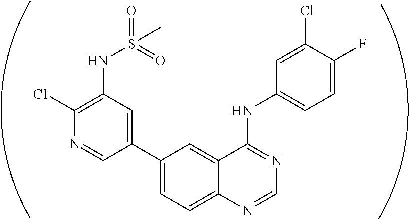

with a quinazoline scaffold was designed, synthesized and characterized as one of the most potent promising inhibitors dual inhibitors of EGFR and PI3K (see, e.g., Examples and Tables I and II).

Accordingly, the present invention further provides methods for treating cancers associated with BRAF and KRAS activity through administration of therapeutic amounts of compound MTX-211 to a subject suffering from cancer. The methods are not limited to a particular type of cancer. In some embodiments, the cancer is any cancer associated with mutated BRAF and KRAS. In some embodiments, the cancer is selected from colorectal cancer, pancreatic, melanoma, and non-small cell lung cancer. In some embodiments, the compound is co-administered with one or more anticancer agents. In some embodiments, the anticancer agent is a MAPK pathway inhibitor (e.g., a BRAF inhibitor (e.g., vemurafenib, LY3009120, Dabrafenib, LGX818) (e.g., a MEK inhibitor (e.g., CH5126766/RO5126766, trametinib, MEK162, PDO325901) (e.g., an ERK inhibitor (e.g., SCH772984)).

Moreover, the present invention provides methods for inhibiting EGFR and PI3K activity in cells through exposing such cells to one or more of the quinazoline compounds of the present invention. In some embodiments, the quinazoline compound is compound MTX-211. In some embodiments, the cells are simultaneously exposed to a MAPK pathway inhibitor (e.g., a BRAF inhibitor (e.g., vemurafenib, LY3009120, Dabrafenib, LGX818) (e.g., a MEK inhibitor (e.g., CH5126766/RO5126766, trametinib, MEK162, PDO325901) (e.g., an ERK inhibitor (e.g., SCH772984)).

### BRIEF DESCRIPTION OF DRAWINGS

FIG. 1 shows that MTX-211 is potent and highly selective against ERBB and PI3K family members.

FIG. 2 shows that MTX-211 has favorable metabolic stability.

FIG. 3 shows that MTX-211 has favorable bioactivity.

FIG. 4 shows that MTX-211 modulates cellular EGFR and PI3K pathway signaling.

FIG. 5 shows that single agent activity of MTX-211 against HCT-116 (KRAS^{mt}) and RKO (BRAF^{mt}) tumors.

FIG. 6 shows that daily oral dosing of MTX-211 of 100 mg/kg is well tolerated.

FIG. 7A shows that MTX-211 and trametinib are synergistic in vitro in KRAS^{mt} and BRAF^{mt} models.

FIG. 7B shows synergy of MTX-211 and trametinib in RKO colony-forming assays; results obtained from clonogenic assay analysis.

FIG. 8 shows synergy of MTX-211 and trametinib in colony-forming assays.

FIG. 9 shows MTX-211/trametinib combination leads to potentiation of signaling events.

FIG. 10 shows the impact of MTX-211/trametinib combination on survival (A, B, C); Ki-67 Expression (D), and Activation of Critical Signaling Molecules (E).

FIG. 11 shows effect of MTX-211/trametinib on KRAS mutant cell lines; and demonstrates in vitro synergy between MTX-211 and the MEK inhibitor trametinib against KRAS mutant (HCT-116) colorectal cancer cells (data optained from cell viability assays).

FIG. 12 shows effect of MTX-211/trametinib on BRAF mutant cell lines.

FIG. 13 shows effect of MTX-211/trametinib on BRAF mutant cell lines.

FIG. 14 shows effect of MTX-211/trametinib on BRAF mutant cell lines; and demonstrates in vitro synergy between MTX-211 and the MEK inhibitor trametinib against BRAF mutant (RKO) colorectal cancer cells (data optained from cell viability assays).

FIG. 15 shows effect of MTX-211/vemurafenib on BRAF mutant cell lines.

FIG. 16 shows effect of MTX-211/vemurafenib on BRAF mutant cell lines.

FIG. 17 shows effect of MTX-211/vemurafenib on BRAF mutant cell lines.

FIG. 18 shows effect of MTX-211/trametinib on BRAF tumor burden.

FIG. 19A shows effect of MTX-211/vemurafenib in RKO cell lines, and MTX-211/LY3009120 in RKO cell lines.

FIG. 19B shows effect of MTX-211/Dabrafenib in RKO cell lines, and MTX-211/LGX818 in RKO cell lines.

FIG. 20A shows effect of MTX-211/Cobimetinib in RKO cell lines, and MTX-211/MEK162 in RKO cell lines.

FIG. 20B shows effect of MTX-211/PD0325901 in RKO cell lines, MTX-211/AZD6244 in RKO cell lines, and MTX-211/R05126766 in RKO cell lines.

FIG. 21 shows effect of MTX-211/SCH772984 in RKO cell lines.

FIG. 22 shows in vivo potentiation of MTX-211 efficacy when combined with MEK inhibitors.

### DEFINITIONS

The term “anticancer agent” as used herein, refer to any therapeutic agents (e.g., chemotherapeutic compounds and/or molecular therapeutic compounds), antisense therapies, radiation therapies, or surgical interventions, used in the treatment of hyperproliferative diseases such as cancer (e.g., in mammals, e.g., in humans).

The term “prodrug” as used herein, refers to a pharmacologically inactive derivative of a parent “drug” molecule that requires biotransformation (e.g., either spontaneous or enzymatic) within the target physiological system to release, or to convert (e.g., enzymatically, physiologically, mechanically, electromagnetically) the prodrug into the active drug. Prodrugs are designed to overcome problems associated with stability, water solubility, toxicity, lack of specificity, or limited bioavailability. Exemplary prodrugs comprise an active drug molecule itself and a chemical masking group (e.g., a group that reversibly suppresses the activity of the drug). Some prodrugs are variations or derivatives of compounds that have groups cleavable under metabolic conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of water solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol) or esters prepared by reaction of parent alcohol with a suitable carboxylic acid, (e.g., an amino acid), amides prepared by reaction of the parent acid compound with an amine, basic groups reacted to form an acylated base derivative (e.g., a lower alkylamide), or phosphorus-containing derivatives, e.g., phosphate, phosphonate, and phosphoramidate esters, including cyclic phosphate, phosphonate, and phosphoramidate (see, e.g., U.S. Patent Application Publication No. 2007/0249564 A1; herein incorporated by reference in its entirety).

The term “pharmaceutically acceptable salt” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “solvate” as used herein, refers to the physical association of a compound of the invention with one or more solvent molecules, whether organic or inorganic. This physical association often includes hydrogen bonding. In certain instances, the solvate is capable of isolation, for example, when one or more solvate molecules are incorporated in the crystal lattice of the crystalline solid. “Solvate” encompasses both solution-phase and isolable solvates. Exemplary solvates include hydrates, ethanolates, and methanolates.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a quinazoline compound of the invention), an animal or a cell within an animal more susceptible, or more responsive, to the biological effects (e.g., promotion or retardation of an aspect of cellular function including, but not limited to, cell division, cell growth, proliferation, invasion, angiogenesis, necrosis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least 300%, at least about 350%, at least about 400%, at least about 450%, or at least about 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, non-limiting examples of which include, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

### DETAILED DESCRIPTION OF THE INVENTION

The present invention addresses the need for improved methods for treating cancers associated with both KRAS and BRAF mutations. Indeed, experiments conducted during the course of developing embodiments for the present invention designed a new class of potent small-molecules having a quinazoline structure which function as dual inhibitors EGFR and PI3K. Such experiments further determined that a combination of such dual EGFR/PI3K inhibitors with MAPK pathway inhibitors (e.g., trametinib) resulted in a synergistic effect for treating cancers associated with both KRAS and BRAF mutations.

As such, the present invention provides a new class of small-molecules having a quinazoline structure which function as dual inhibitors EGFR and PI3K, and their use as therapeutics for the treatment of cancer and other diseases (e.g., in combination with MAPK pathway inhibitors).

Accordingly, the present invention contemplates that exposure of animals (e.g., humans) suffering from cancer (e.g., cancer associated with KRAS and BRAF mutations) (e.g., and/or cancer related disorders) to therapeutically effective amounts of drug(s) having a quinazoline structure (e.g., small molecules having a quinazoline structure) that inhibit the activity of both EGFR and PI3K will inhibit the growth of such cancer cells or supporting cells outright and/or render such cells as a population more susceptible to the cell death-inducing activity of cancer therapeutic drugs or radiation therapies.

Moreover, the present invention contemplates that such a therapeutic effect is enhanced (e.g., synergized) through combination treatment (e.g., simultaneous, non-simultaneous) with MAPK pathway inhibitors. Indeed, the present invention contemplates that dual inhibitors of EGFR and PI3K activity satisfy an unmet need for the treatment of multiple cancer types, either when administered as monotherapy to induce cell growth inhibition, apoptosis and/or cell cycle arrest in cancer cells, or when administered in a temporal relationship with additional agent(s), such as other cell death-inducing or cell cycle disrupting cancer therapeutic drugs (e.g., MAPK pathway inhibitors) or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In a particular embodiment, quinazoline compounds encompassed within Formula I are provided:

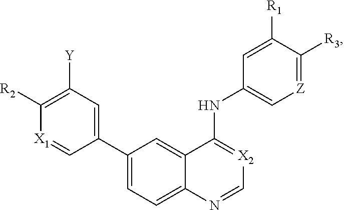

including pharmaceutically acceptable salts, solvates, and/or prodrugs thereof.

Formula I is not limited to a particular chemical moiety for R1, R2, R3, X1, X2, Y or Z. In some embodiments, the particular chemical moiety for R1, R2, R3, X1, X2, Y or Z independently include any chemical moiety that permits the resulting compound to inhibit both EGFR and PI3K activity. In some embodiments, the particular chemical moiety for R1, R2, R3, X1, X2, Y or Z independently include any chemical moiety that permits the resulting compound to effectively treat cancers associated with KRAS and BRAF mutations when administered alone or in combination with a MAPK pathway inhibitor (e.g., a BRAF inhibitor (e.g., vemurafenib, LY3009120, Dabrafenib, LGX818) (e.g., a MEK inhibitor (e.g., CH5126766/RO5126766, trametinib, MEK162, PDO325901) (e.g., an ERK inhibitor (e.g., SCH772984)).

In some embodiments, R1 is hydrogen, halogen (e.g., Chlorine, Fluorine), or

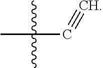 [SMILES: *C#C]

In some embodiments, R2 is hydrogen, halogen (e.g., Chlorine, Fluorine), or methoxy (e.g.,

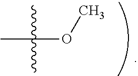 [SMILES: *OC]

In some embodiments, R3 is hydrogen, halogen (e.g., Chlorine, Fluorine), methoxy or pyridin-2-ylmethoxy (e.g.,

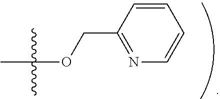 [SMILES: *OCc1ccccn1]

In some embodiments, X1 is Nitrogen or CH(e.g.,

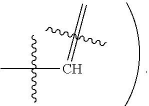 [SMILES: *C=*]

In some embodiments, X2 is Nitrogen or CH-Nitrile(e.g.,

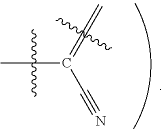 [SMILES: *C(=*)C#N]

In some embodiments, Y is hydrogen, NHSO₂CH₃, or NHSO₂CH₂CH₂N(CH₃)₂ e.g.,

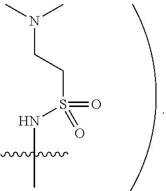 [SMILES: *NS(=O)(=O)CCN(C)C]

In some embodiments, Z is Nitrogen or CH (e.g.,

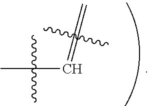 [SMILES: *C=*]

In some embodiments, the following compounds are contemplated for Formula I:

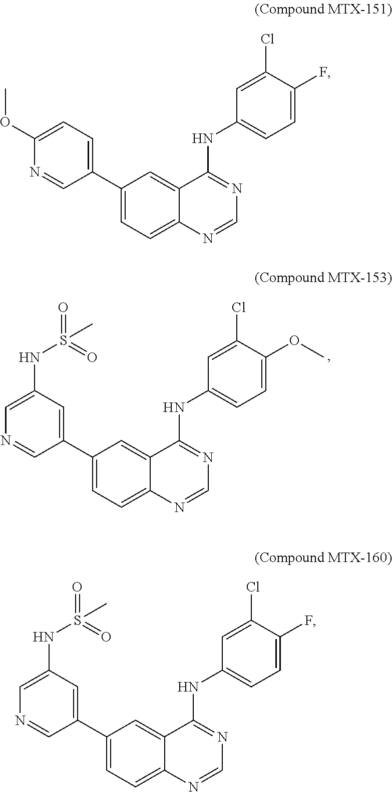 [SMILES: COc1ccc(-c2ccc3ncnc(Nc4ccc(F)c(Cl)c4)c3c2)cn1 ; COc1ccc(Nc2ncnc3ccc(-c4cncc(NS(C)(=O)=O)c4)cc23)cc1Cl ; CS(=O)(=O)Nc1cncc(-c2ccc3ncnc(Nc4ccc(F)c(Cl)c4)c3c2)c1]

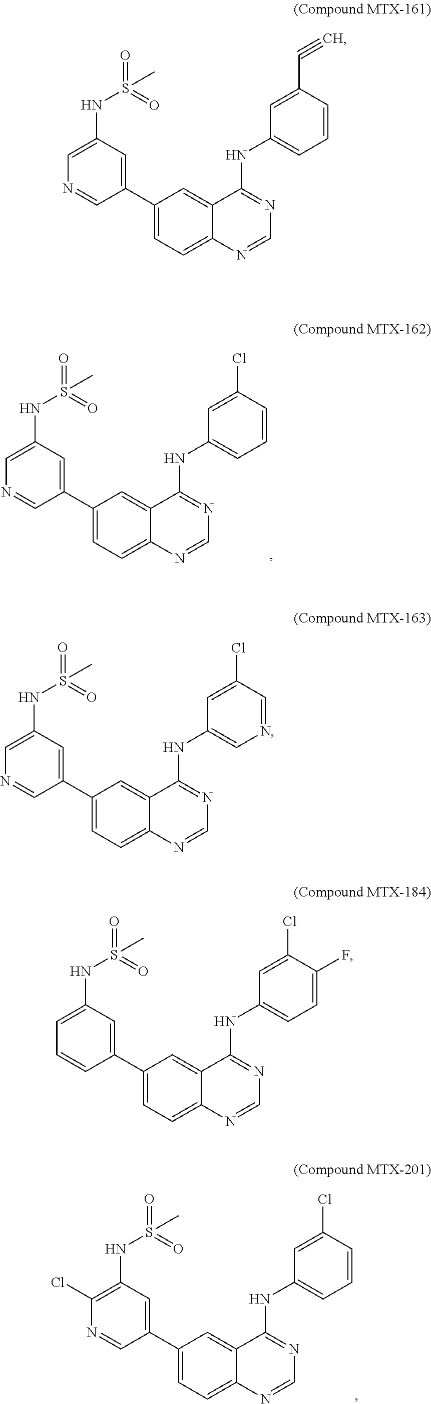 [SMILES: C#Cc1cccc(Nc2ncnc3ccc(-c4cncc(NS(C)(=O)=O)c4)cc23)c1 ; CS(=O)(=O)Nc1cncc(-c2ccc3ncnc(Nc4cccc(Cl)c4)c3c2)c1 ; CS(=O)(=O)Nc1cncc(-c2ccc3ncnc(Nc4cncc(Cl)c4)c3c2)c1 ; CS(=O)(=O)Nc1cccc(-c2ccc3ncnc(Nc4ccc(F)c(Cl)c4)c3c2)c1 ; CS(=O)(=O)Nc1cc(-c2ccc3ncnc(Nc4cccc(Cl)c4)c3c2)cnc1Cl]

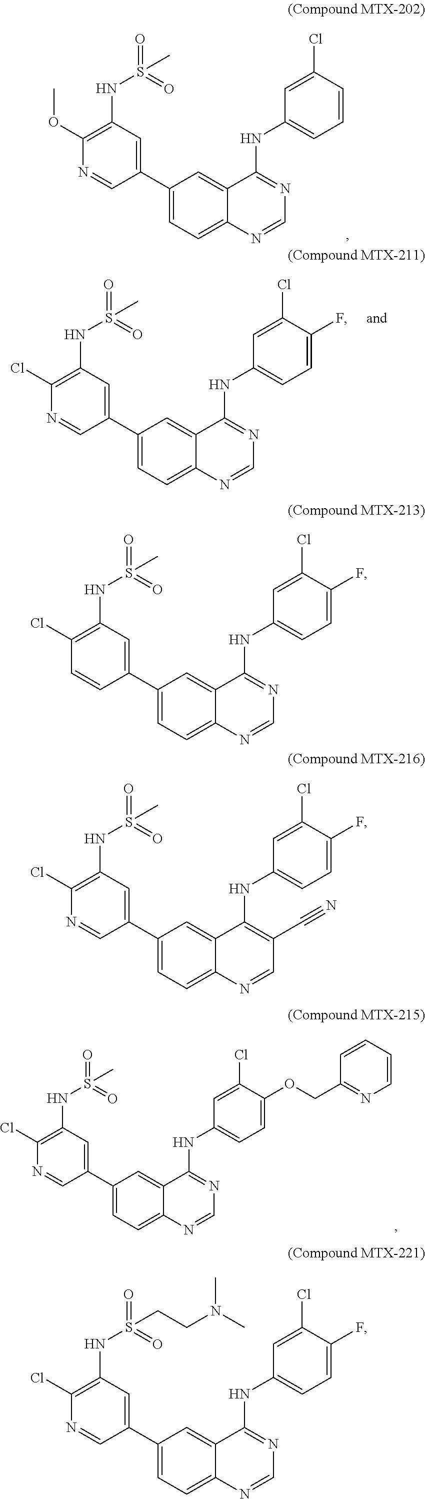 [SMILES: COc1ncc(-c2ccc3ncnc(Nc4cccc(Cl)c4)c3c2)cc1NS(C)(=O)=O ; CS(=O)(=O)Nc1cc(-c2ccc3ncnc(Nc4ccc(F)c(Cl)c4)c3c2)cnc1Cl ; CS(=O)(=O)Nc1cc(-c2ccc3ncnc(Nc4ccc(F)c(Cl)c4)c3c2)ccc1Cl ; CS(=O)(=O)Nc1cc(-c2ccc3ncc(C#N)c(Nc4ccc(F)c(Cl)c4)c3c2)cnc1Cl ; CS(=O)(=O)Nc1cc(-c2ccc3ncnc(Nc4ccc(OCc5ccccn5)c(Cl)c4)c3c2)cnc1Cl ; CN(C)CCS(=O)(=O)Nc1cc(-c2ccc3ncnc(Nc4ccc(F)c(Cl)c4)c3c2)cnc1Cl]

or a pharmaceutically acceptable salt, solvate, or prodrug thereof.

An important aspect of the present invention is that compounds of the invention induce cell cycle arrest and/or apoptosis and also potentiate the induction of cell cycle arrest and/or apoptosis either alone or in response to additional apoptosis induction signals. Therefore, it is contemplated that these compounds sensitize cells to induction of cell cycle arrest and/or apoptosis, including cells that are resistant to such inducing stimuli. The dual EGFR and PI3K inhibitors of the present invention (e.g., quinazoline compounds) can be used to induce apoptosis in any disorder that can be treated, ameliorated, or prevented by the induction of apoptosis.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, pancreatic cancer, breast cancer, prostate cancer, lymphoma, skin cancer, colon cancer, melanoma, malignant melanoma, ovarian cancer, brain cancer, primary brain carcinoma, head and neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic.

In other embodiments, the cancer cells being treated are resistant to anticancer agents.

In other embodiments, the disorder is any disorder having cells having BRAF and KRAS activity.

Some embodiments of the present invention provide methods for administering an effective amount of a compound of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies). In a particular embodiment, the additional therapeutic agent(s) is an anticancer agent.

In some embodiments, the additional therapeutic agent is a MAPK pathway inhibitor. Examples of MAPK pathway inhibitors include, but are not limited to, a BRAF inhibitor (e.g., vemurafenib, LY3009120, Dabrafenib, LGX818), a MEK inhibitor (e.g., CH5126766/RO5126766, trametinib, MEK162, PDO325901), and/or an ERK inhibitor (e.g., SCH772984)).

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 3 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 3

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4 H-pyrazolo[3,4-d]pyrimidin-4- | | Research Triangle Park, |
| one monosodium salt) | | NC |
| Altretamine | Hexalen | US Bioscience, West |
| (N,N,N′,N′,N″,N″,-hexamethyl-1,3,5-triazine- | | Conshohocken, PA |
| 2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain | | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] benzoic | | |
| acid) | | |
| bexarotene gel | Targretin | Ligand Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, 2- | R. W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers Squibb |
| (2-[bis(2-chloroethyl)amino] tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar |
| ((8S-cis)-8-acetyl-10-[(3-amino-2,3,6-trideoxy- | | Pharmaceuticals, Inc., |
| á-L-lyxo-hexopyranosyl)oxy]-7,8,9,10- | | Boulder, CO |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, |
| ((1 S,3 S)-3-Acetyl-1,2,3,4,6,11-hexahydro- | | NJ |
| 3,5,12-trihydroxy-10-methoxy-6,11-dioxo-1- | | |
| naphthacenyl 3-amino-2,3,6-trideoxy-(alpha)- | | |
| L-lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one | | |
| 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin | Pharmacia & Upjohn |
| | PFS Intravenous | Company |
| | injection | |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| | | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3-(p- | | |
| isothiocyanatophenyl)-propyl]-[N-[2- | | |
| bis(carboxymethyl)amino]-2-methyl)- | | |
| ethyl]glycine) | | |
| Idarubicin | Idamycin | Pharmacia & Upjohn |
| (5,12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4- | | Company |
| piperidinopiperidino)carbonyloxy]-1H-pyrano[3′,4′:6,7] | | |
| indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., Seattle, |
| (L-Glutamic acid, N[4[[(2amino-5-formyl- | Leucovorin | WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo | | Foundation, Titusville, |
| [2,1-b] thiazole monohydrochloride | | NJ |
| C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asia Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7- | | Exton, Pa |
| one) | | |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,O′] platinum) | | |
| Paclitaxel | TAXOL | Bristol-Myers Squibb |
| (5β, 20-Epoxy-1,2a, 4,7β, 10β, 13a- | | |
| hexahydroxytax-11-en-9-one 4,10-diacetate 2- | | |
| benzoate 13-ester with (2R,3 S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl) 11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and | | |
| b)-D-glucopyranose and 220 mg citric acid | | |
| anhydrous) | | |
| Talc | Sclerosol | Bryan, Corp., Woburn, |
| (Mg₃Si₄O₁₀(OH)₂) | | MA |
| Tamoxifen | Nolvadex | AstraZeneca |
| ((Z)2-[4-(1,2-diphenyl-1-buteny1) phenoxy]- | | Pharmaceuticals |
| N,N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex Corporation |
| (Aziridine,1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′:6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering a compound of the invention with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Ser. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to an animal is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

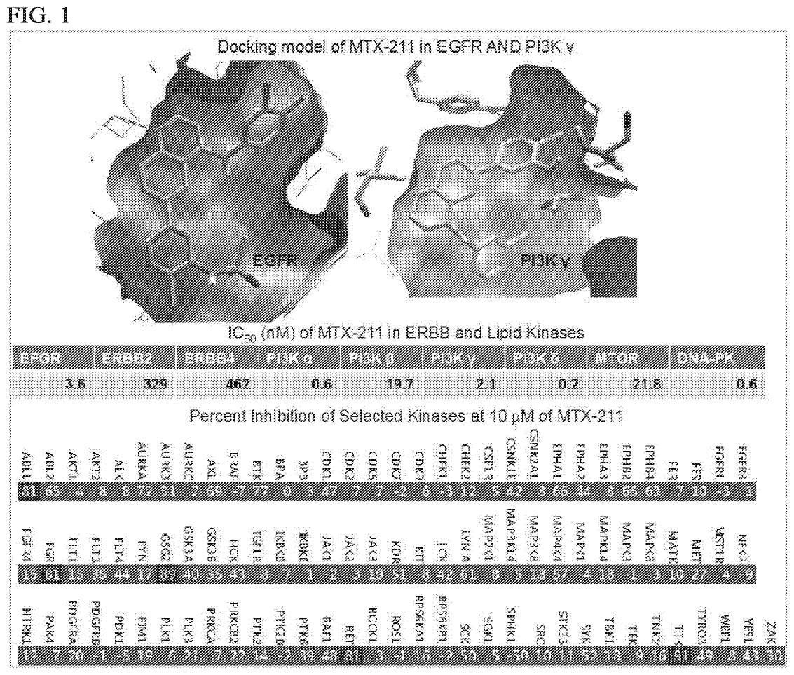

FIG. 1 shows that the compounds of the present invention are potent and highly selective against ERBB and PI3K family members.

Table 1 show IC50 values for specific compounds of the present invention for inhibiting EGFR and PI3K.

TABLE 1

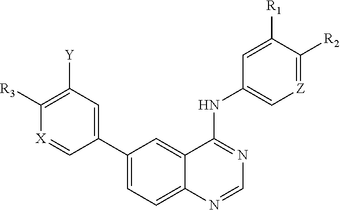

| [symbol] | [symbol] | [symbol] | [symbol] | [symbol] | X | Y | Z | [symbol] | [symbol] |
|---|---|---|---|---|---|---|---|---|---|
| 151 | 1 | Cl | F | OCH₃ | N | H | CH | — | 20 |
| 153 | 2 | Cl | OCH₃ | H | N | NHSO₂CH₃ | CH | 26 | 342 |
| 160 | 3 | Cl | F | H | N | NHSO₂CH₃ | CH | 26.5 | 3.7 |
| 161 | 3 | CCH | H | H | N | NHSO₂CH₃ | CH | 21.4 | 9.0 |
| 162 | 3 | Cl | H | H | N | NHSO₂CH₃ | CH | 24.4 | 1.7 |
| 163 | 3 | Cl | H | H | N | NHSO₂CH₃ | N | 11 | 19 |
| 184 | 3 | Cl | F | H | CH | NHSO₂CH₃ | CH | 1200 | 1.9 |
| 201 | 4 | Cl | H | Cl | N | NHSO₂CH₃ | CH | 0.36 | 2.5 |
| 202 | 4 | Cl | H | OCH₃ | N | NHSO₂CH₃ | CH | 3.2 | 19 |
| 211 | 5 | Cl | F | Cl | N | NHSO₂CH₃ | CH | 0.57 | 3.6 |
| 213 | 5 | Cl | F | Cl | CH | NHSO₂CH₃ | CH | 304 | 8.4 |

[symbol]

indicates data missing or illegible when filed

Table 2 shows mouse and human microsomal stability for specific compounds of the present invention.

TABLE 2

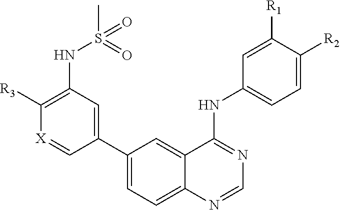

| [symbol] | R1 | R2 | R3 | [symbol] | [symbol] | [symbol] | [symbol] | [symbol] |
|---|---|---|---|---|---|---|---|---|
| 160 | Cl | F | H | N | 26.5 | 3.7 | 9.2 | >60 |
| 161 | CCH | H | H | N | 21.4 | 9.0 | 5.7 | 44 |
| 162 | Cl | H | H | N | 24.4 | 1.7 | 7.5 | >60 |
| 184 | Cl | F | H | CH | 1200 | 1.9 | 23 | >60 |
| 201 | Cl | H | Cl | N | 0.36 | 2.5 | 15 | 49 |
| 211 | Cl | F | Cl | N | 0.57 | 3.6 | >60 | >60 |
| 213 | Cl | F | Cl | CH | 300 | 2.2 | >60 | >60 |

[symbol]

indicates data missing or illegible when filed

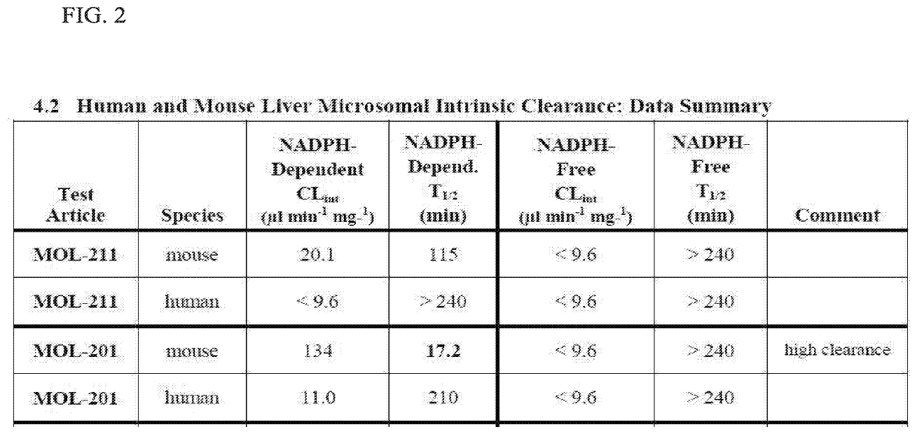

FIG. 2 shows that MTX-211 has favorable metabolic stability.

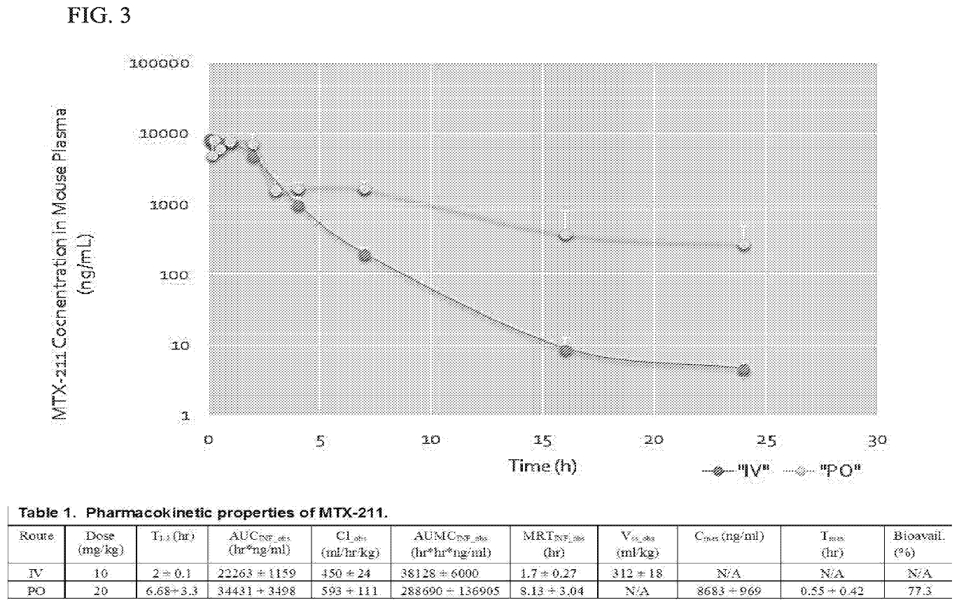

FIG. 3 shows that MTX-211 has favorable bioactivity.

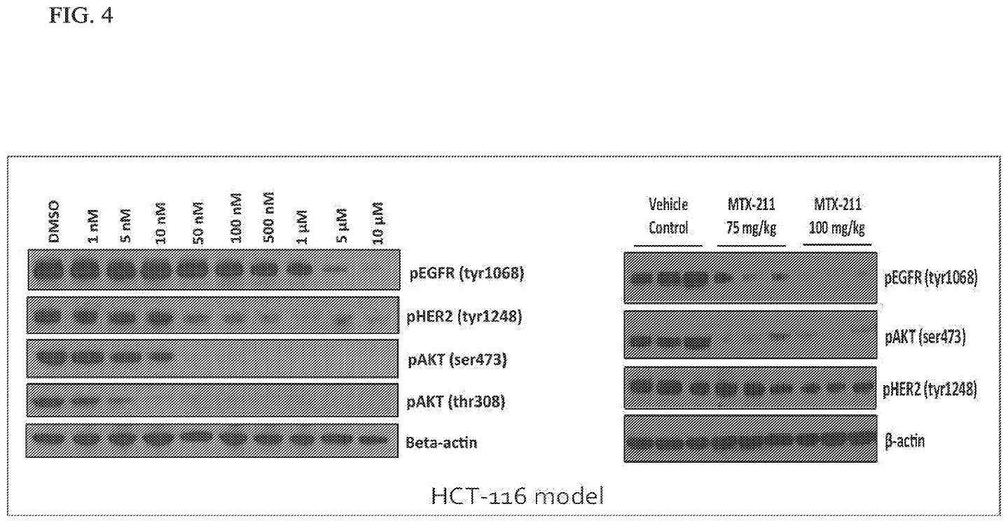

FIG. 4 shows that MTX-211 modulates cellular EGFR and PI3K pathway signaling.

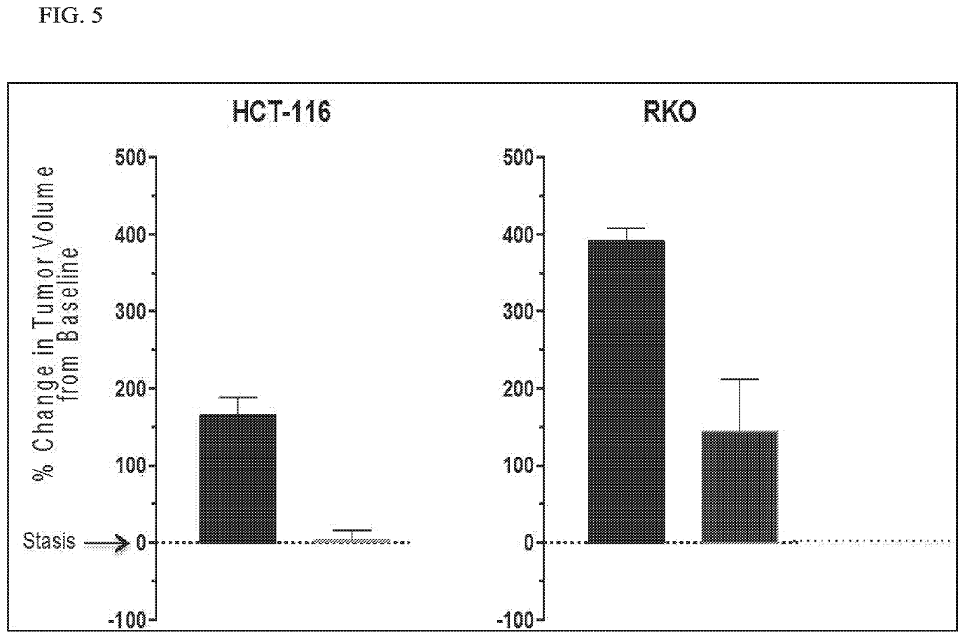

FIG. 5 shows that single agent activity of MTX-211 against HCT-116 (KRAS^{mt}) and RKO (BRAF^{mt}) tumors.

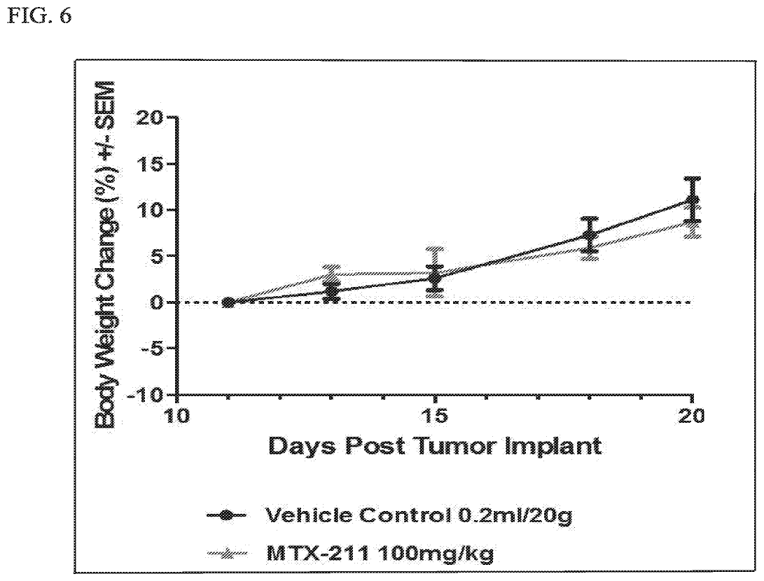

FIG. 6 shows that daily oral dosing of MTX-211 of 100 mg/kg is well tolerated.

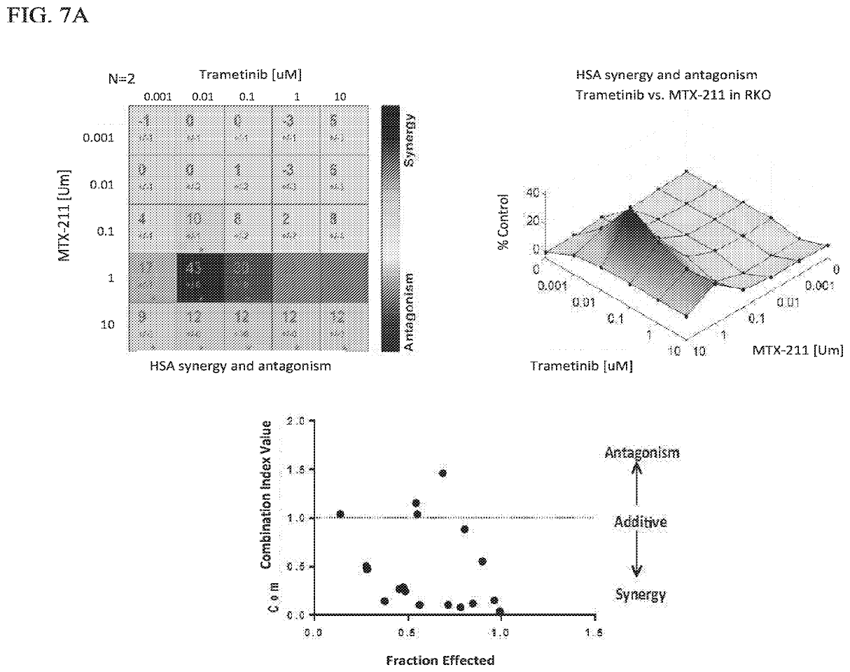

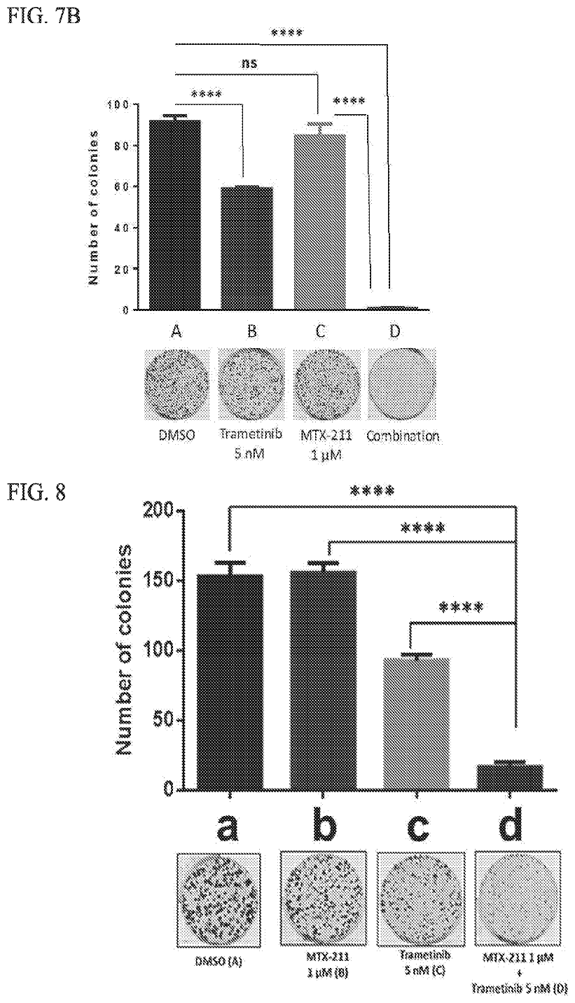

FIG. 7A shows that MTX-211 and trametinib are synergistic in vitro in KRAS^{mt} and BRAF^{mt} models.

FIG. 7B shows synergy of MTX-211 and trametinib in RKO colony-forming assays; results obtained from clonogenic assay analysis.

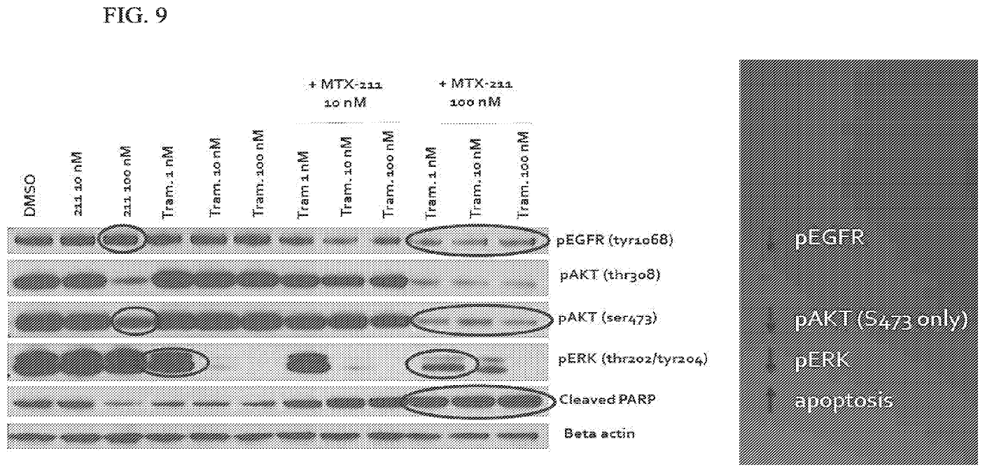

FIG. 8 shows synergy of MTX-211 and trametinib in colony-forming assays.

FIG. 9 shows MTX-211/trametinib combination leads to potentiation of signaling events.

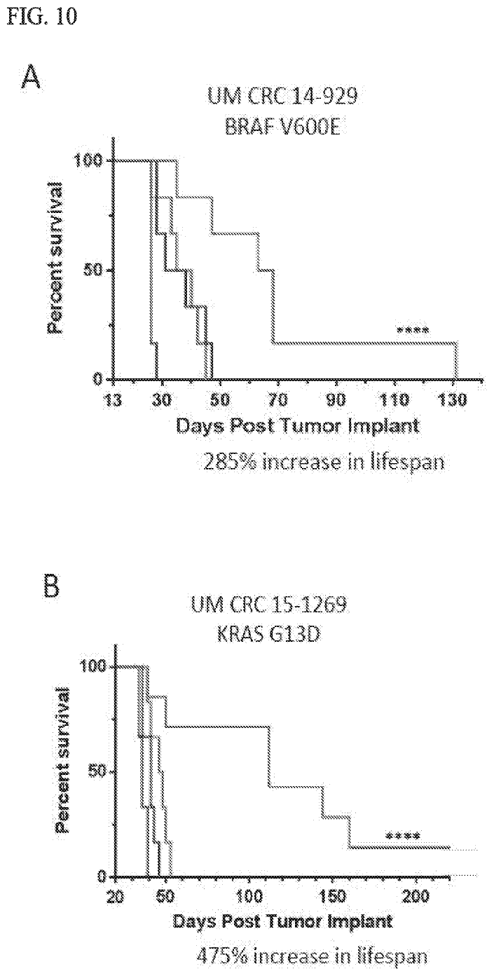

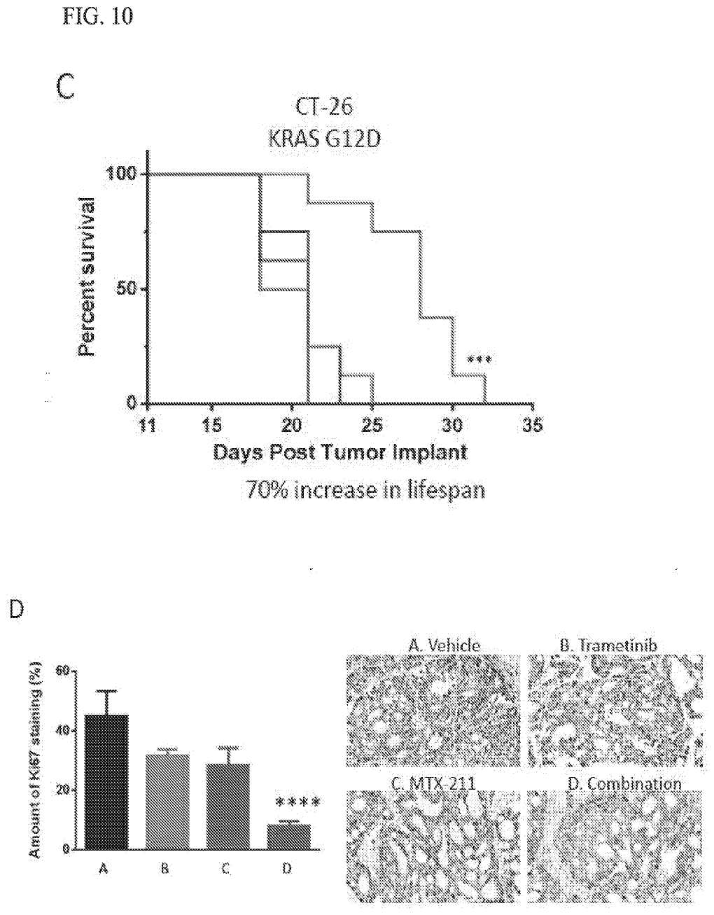

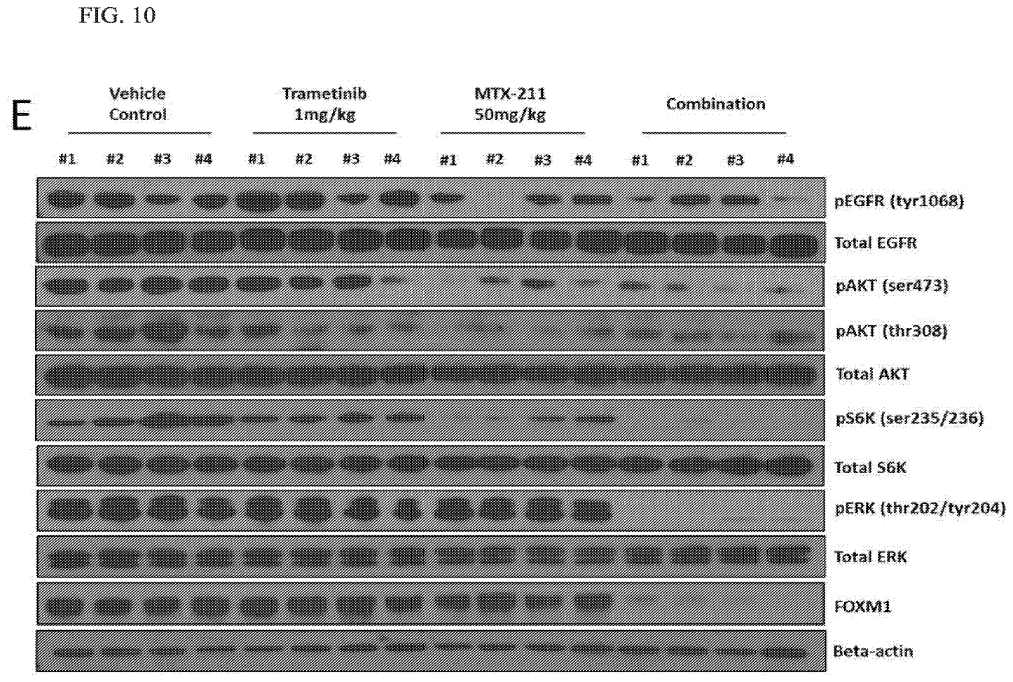

FIG. 10 shows the impact of MTX-211/trametinib combination on survival (A, B, C); Ki-67 Expression (D), and Activation of Critical Signaling Molecules (E).

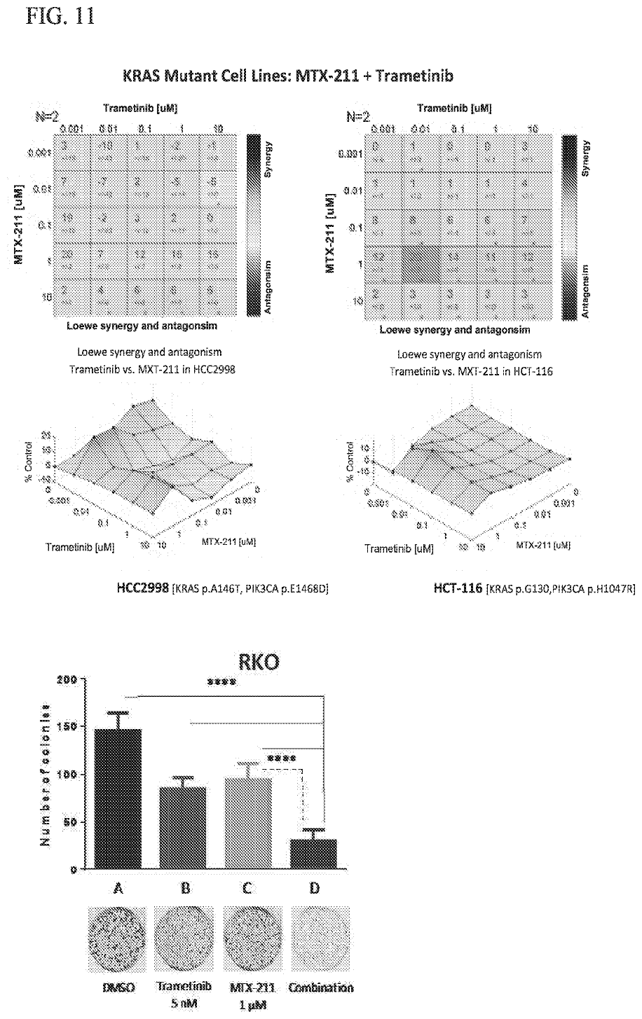

FIG. 11 shows effect of MTX-211/trametinib on KRAS mutant cell lines; and demonstrates in vitro synergy between MTX-211 and the MEK inhibitor trametinib against KRAS mutant (HCT-116) colorectal cancer cells (data optained from cell viability assays).

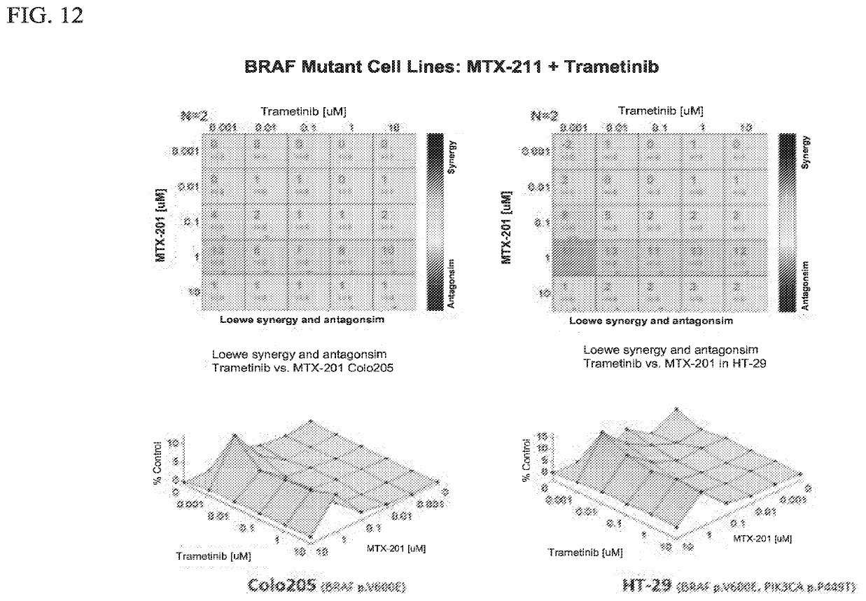

FIG. 12 shows effect of MTX-211/trametinib on BRAF mutant cell lines.

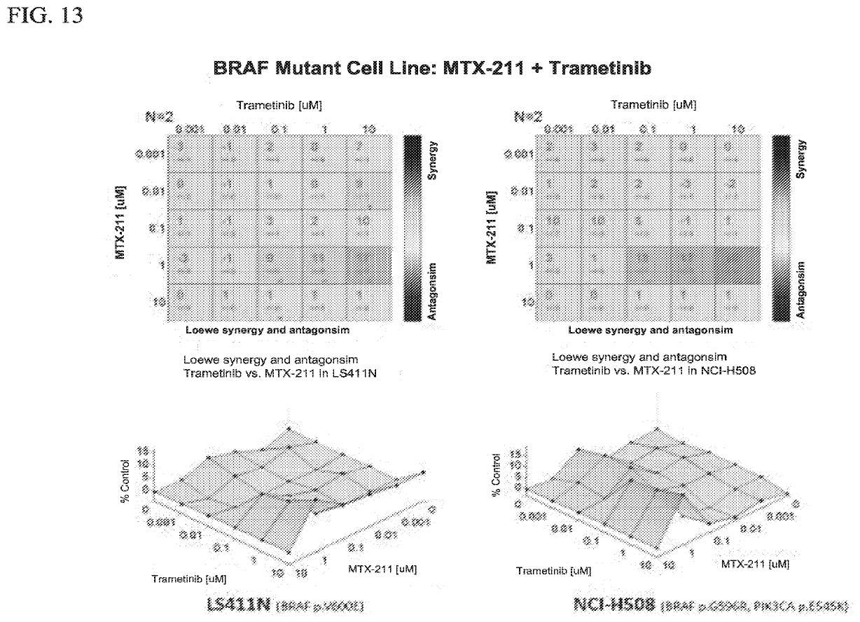

FIG. 13 shows effect of MTX-211/trametinib on BRAF mutant cell lines.

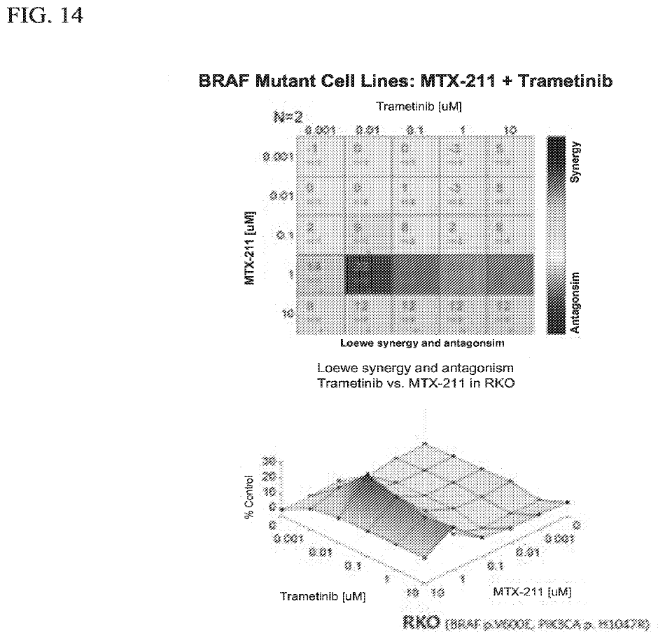

FIG. 14 shows effect of MTX-211/trametinib on BRAF mutant cell lines; and demonstrates in vitro synergy between MTX-211 and the MEK inhibitor trametinib against BRAF mutant (RKO) colorectal cancer cells (data optained from cell viability assays).

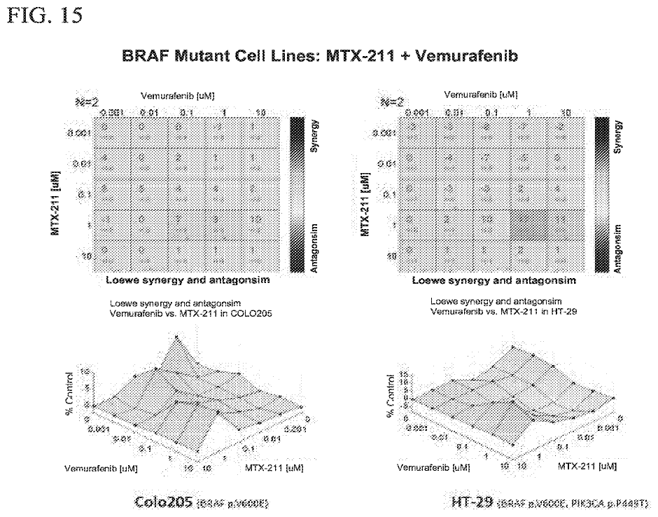

FIG. 15 shows effect of MTX-211/vemurafenib on BRAF mutant cell lines.

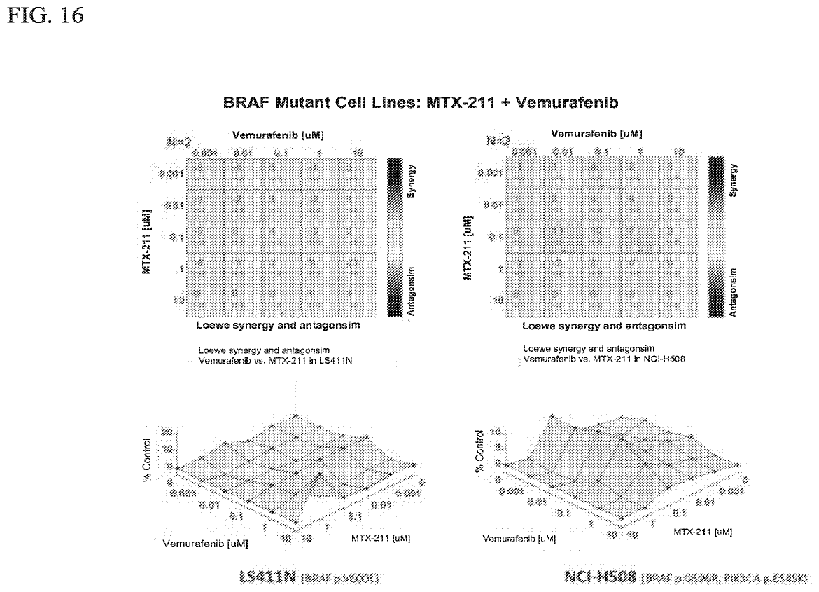

FIG. 16 shows effect of MTX-211/vemurafenib on BRAF mutant cell lines.

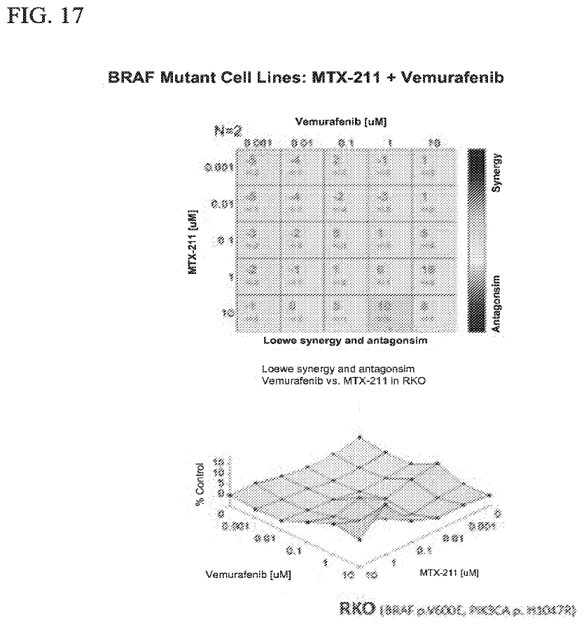

FIG. 17 shows effect of MTX-211/vemurafenib on BRAF mutant cell lines.

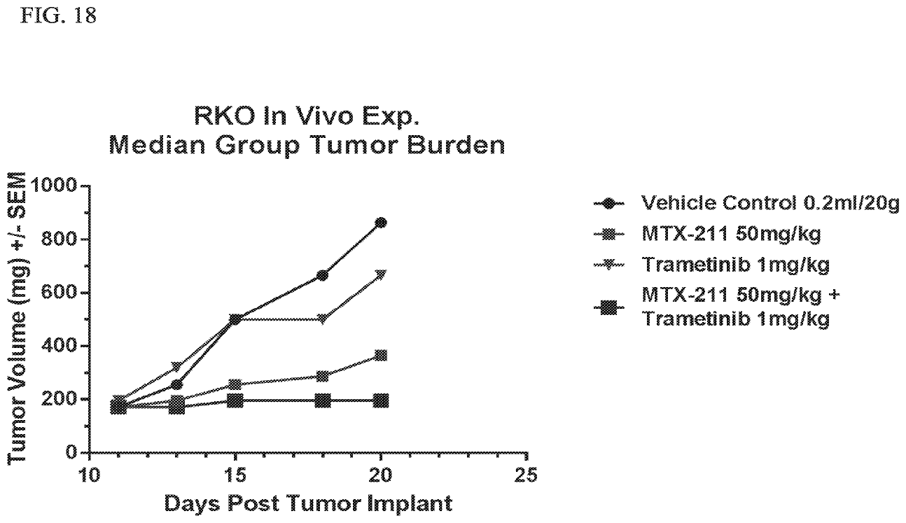

FIG. 18 shows effect of MTX-211/trametinib on BRAF tumor burden.

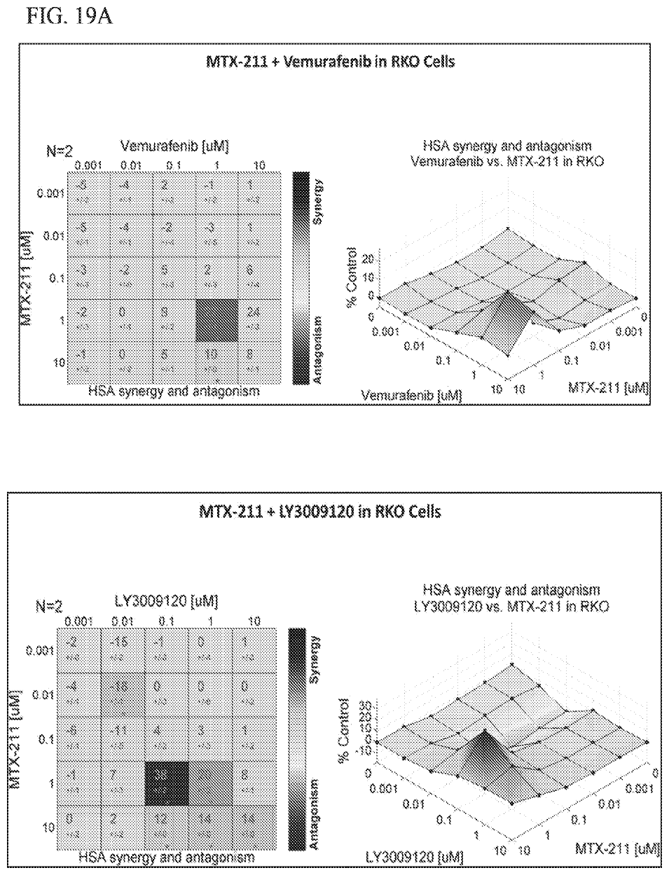

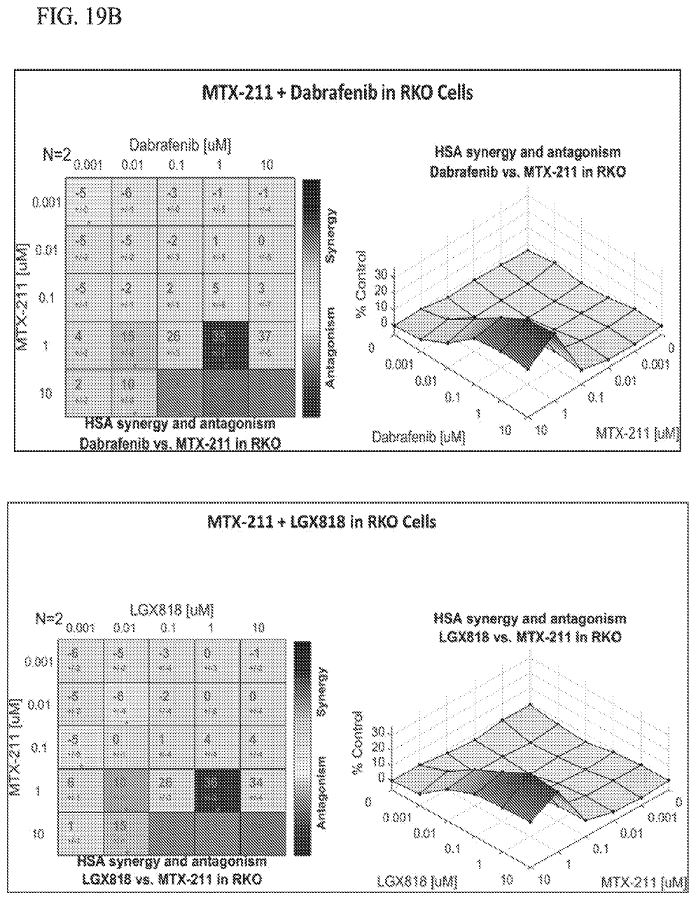

FIG. 19A shows effect of MTX-211/vemurafenib in RKO cell lines, and MTX-211/LY3009120 in RKO cell lines.

FIG. 19B shows effect of MTX-211/Dabrafenib in RKO cell lines, and MTX-211/LGX818 in RKO cell lines.

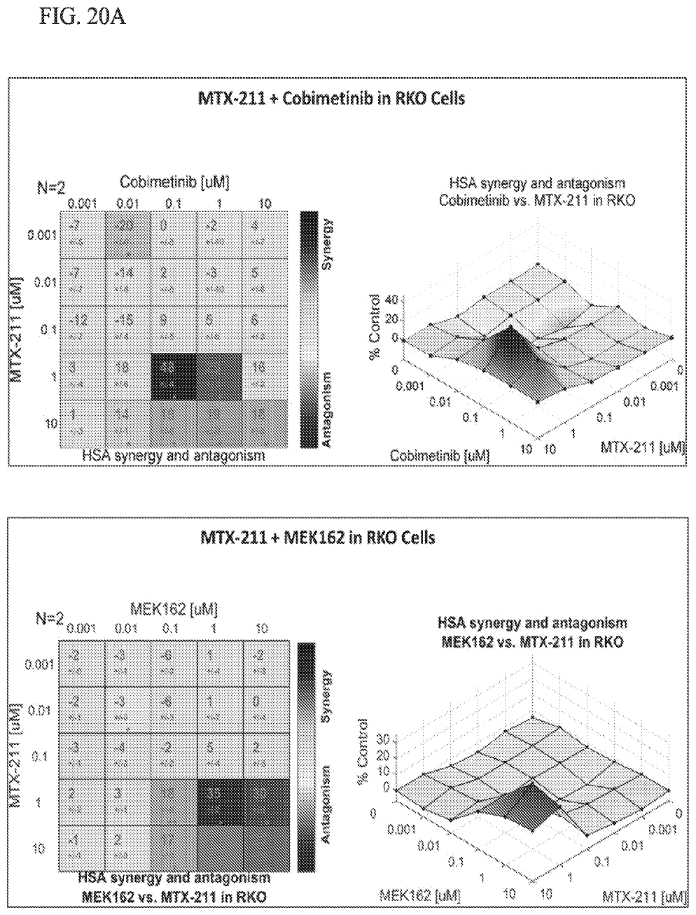

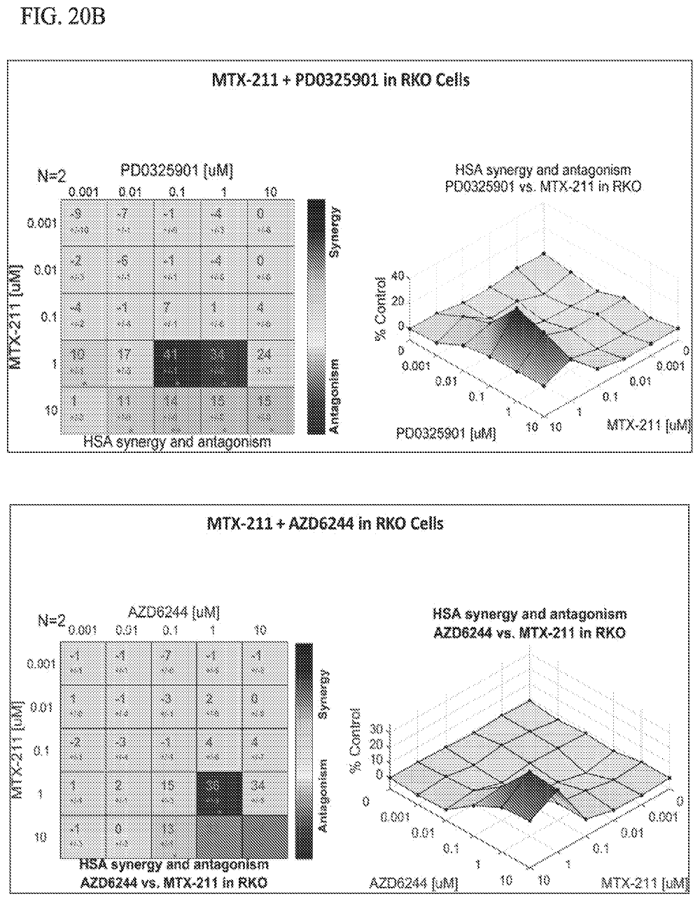

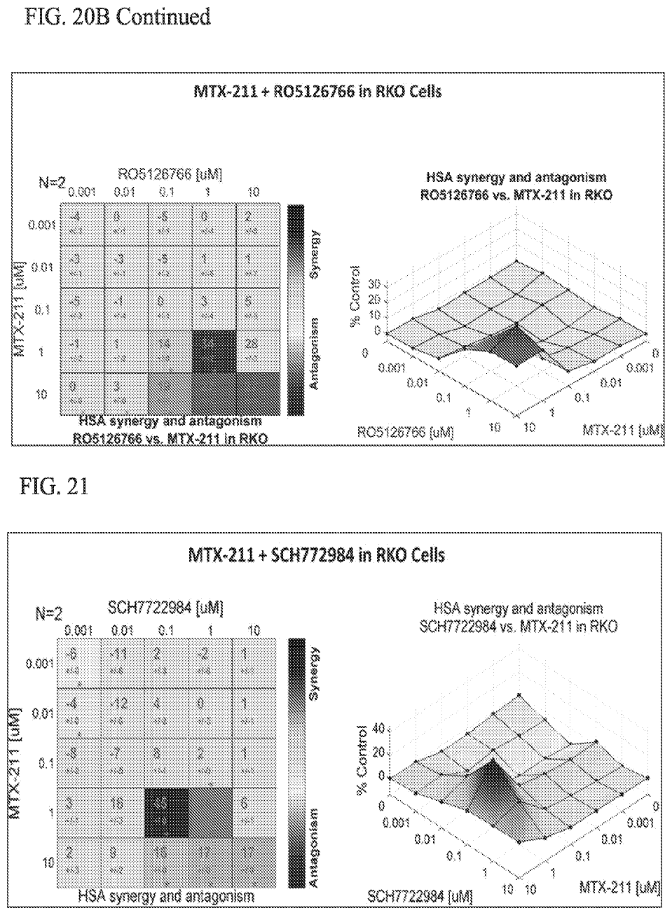

FIG. 20A shows effect of MTX-211/Cobimetinib in RKO cell lines, and MTX-211/MEK162 in RKO cell lines.

FIG. 20B shows effect of MTX-211/PD0325901 in RKO cell lines, MTX-211/AZD6244 in RKO cell lines, and MTX-211/R05126766 in RKO cell lines.

FIG. 21 shows effect of MTX-211/SCH772984 in RKO cell lines.

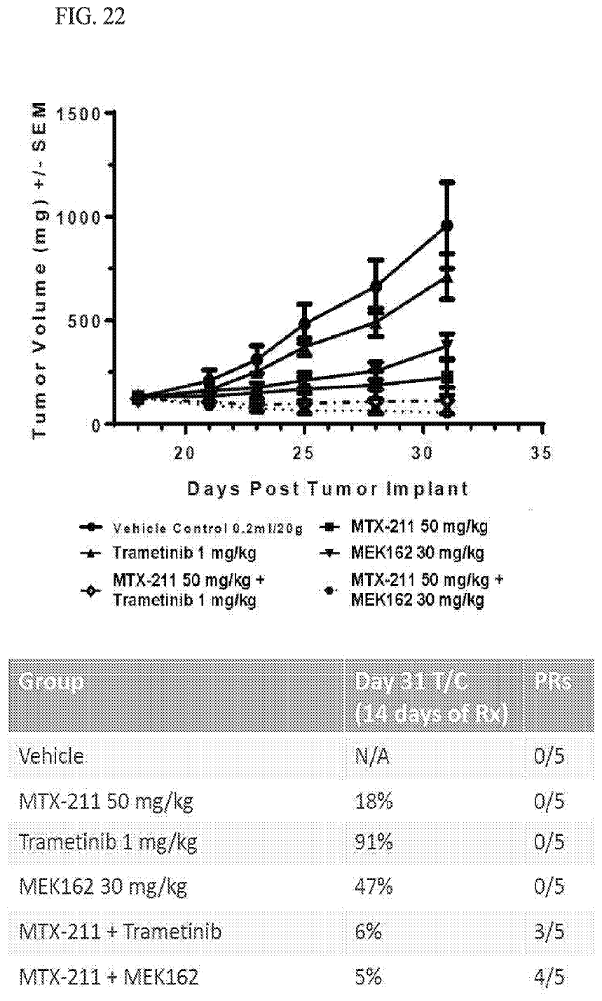

FIG. 22 shows in vivo potentiation of MTX-211 efficacy when combined with MEK inhibitors.

### Example II

This example describes the materials and methods used for Example I.

### Cell Culture and Inhibitors

HCT-116 and RKO cells were obtained from the American Type Culture Collection ATCC). HCT-116 cells were maintained in McCoy's 5A media (Invitrogen) supplemented with 10% FBS (HyClone), 1% GlutaMax, (Invitrogen) and 1% Penicillin Streptomycin (Invitrogen). RKO cells were maintained in EMEM media (Lonza) supplemented with 10% FBS (HyClone), 1% GlutaMax, (Invitrogen) and 1% Penicillin Streptomycin (Invitrogen). All cells were incubated at 37° C. in 5% CO₂. Cell line validation was performed by the University of Michigan DNA Sequencing Core using short tandem repeat analysis.

### Drugs

For cellular studies, drugs were dissolved in DMSO at a concentration of 10 mmol/L and stock solutions were stored at −20° C.

### Cell Viability Assay

For growth inhibition analysis, cells were seeded in whitewalled/clear-bottom tissue culture treated 96-well plates and allowed to adhere for 24 hours followed by addition of growth media containing serial dilutions of MTX-211, MAP kinase pathway inhibitor, or both drugs in combination. Control wells received DMSO at a final concentration of 0.2%. Cells were incubated for 3 days in the continuous presence of drug or DMSO and viability was measured using CellTiter-Glo (Promega). Viability was calculated as a percentage of the DMSO-treated cells. Four replicates were performed for each of the different drug treatment conditions. Data were modeled using a nonlinear regression curve fit with a sigmoidal dose—response using GraphPad Prism 6 (GraphPad Software). Synergy calculations were performed using Combenefit software (Cancer Research UK Cambridge Institute).

### Clonogenic Assay

For each cell line, 500 cells were plated per well into 6-well plates, with six replicates per treatment condition. The cells were allowed to attach overnight. Cells were treated with MTX-211, trametinib, or the combination at the concentrations indicated in the figure legends. Ten days later, the cells were fixed with 10% neutral buffered formalin (NBF) and the stained using 0.1% crystal violet. The colonies were counted using OpenCFU open-access software. Quantification is presented as mean±SEM. In assessing the different treatment conditions, a one-way ANOVA test was used for statistical analysis.

### Western Blots

Cells or tumors were lysed in NP-40 lysis buffer [25 mmolL Tris-HCl (pH 7.6), 150 mmol/L NaCl, 1% Nonidet P-40, 10% glycerol, 1 mmol/L EDTA, 1 mmol/L dithiothreitol, and protease and phosphatase inhibitors], rocked for 30 minutes at 4° C., and centrifuged at 13,200 rpm for 20 minutes at 4° C. Protein concentration was determined by BioRad Protein Assays and lysates were subsequently subjected to SDS gel electrophoresis. Proteins were transferred to polyvinylidene fluoride (PVDF) membranes and probed with primary antibodies recognizing p-EGFR tyr1068), EGFR, p-HER2 (tyr1248), HER2, p-AKT (ser473), p-AKT (thr308), AKT, pERK1/2 (thr202/tyr204), ERK1/2, pS6K (ser235/236), S6K, and cleaved PARP (all from Cell Signaling Technology) and beta actin (Abcam). After incubation with anti-rabbit HRP-linked secondary antibody (Jackson ImmunoResearch Laboratories, Inc.), proteins were detected using chemiluminescence (GE Healthcare).

### Xenograft Studies

For the xenograft studies established from the patient-derived xenograft (PDX) models (UM CRC 14-929 and UM CRC 15-1269), female 6- to 7-week-old NCR nude mice (CrTac:NCr-Foxnlnu from Taconic) were implanted subcutaneously with low-passage PDX tumor fragments (30 mg) into the region of the right axilla. For the xenograft studies established from cell lines (CT-26), cells were injected (1×10⁶ cells per injection) into the flanks of female 6- to 7-week old NCR nude mice. In both cases, the mice were randomized into treatment groups and treatments initiated when tumors reached 100 to 200 mg. MTX-211 and trametinib or MEK162 were administered daily by oral gavage as a solution in 5% dimethyl sulfoxide and 95% polyethylene glycol (MTX-211) and a fine suspension in 0.5% HPMC with 0.2% Tween-80 (trametinib and MEK162), based upon individual animal body weight (0.2 mL/20 g). Subcutaneous tumor volume and body weights were measured two to three times a week. Tumor volumes were calculated by measuring two perpendicular diameters with calipers and using the formula: tumor volume ¼ (length x width²)/2. Percent treated/control (% TC) was calculated by dividing the median treated tumor weight by the median control tumor weight and multiplying by 100 on the last day of treatment. A one-sided unpaired T-test was used to assess differences between the vehicle control and the MTX-211 treated mice. Partial responders (PRs) are defined as having a decrease in tumor volume of >50% when compared to baseline tumor volume at initiation of treatment. For the lifespan assays, the mice were treated daily as indicated until their individual tumor burdens surpassed 1000 mg at which point the mice were euthanized. Increase in lifespan was calculated by dividing the median increase in lifespan (days) by the median survival time of the vehicle control group. A log-rank (Mantel-Cox) test was run to compare the difference in survival between the treatment groups. All procedures related to the handling, care, and treatment of animals were conducted in accordance with University of Michigan's Institutional Animal Care and Use Committee guidelines.

### Immunohistochemisty

Tissues were fixed in 10% NBF, embedded in paraffin, and sectioned in accordance with standard procedures. The Ki67 antibody was obtained from Abcam (ab15580). The slides were scanned using a 3D Histotech Panoramic SCAN II. Images were captured using CaseViewer software. Images were taken with a Nikon E-800 microscope, Olympus DP71 digital camera, and DP Controller software. For quantification of staining, representative images were obtained from the stained slides at ×40 objective magnification for ImmunoRatio analysis. For each treatment condition, five representative fields of view from four individual tumors were analyzed. The images were analyzed using the basic mode in the ImmunoRatio software. Quantification is presented as mean±SEM. In assessing the different treatment conditions, a one-way ANOVA test was used for statistical analysis.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating a cancer in a patient, the method comprising administering a therapeutically effective amount of a combination to the patient in need thereof, the combination comprising: a compound having Formula I: 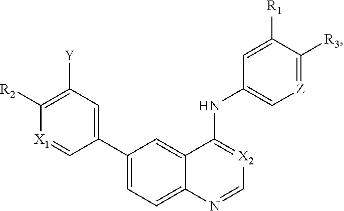
including pharmaceutically acceptable salts, solvates, and/or prodrugs thereof; wherein
R₁ is independently: hydrogen, halogen, or 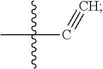 [SMILES: *C#C]
R₂ is independently hydrogen, halogen, or methoxy;
R₃ is independently hydrogen, halogen, methoxy, or pyridin-2-ylmethoxy;
X₁ is independently nitrogen or —CH═;
X₂ is independently nitrogen or —C-nitrile;
Y is independently hydrogen, NHSO₂CH₃, or NHSO₂CH₂CH₂N(CH₃)₂; and
Z is independently nitrogen or —CH═;
and at least one MAPK pathway inhibitor.

2.-8. (canceled)

9. The method of claim 1, wherein the compound of Formula I is selected from the group consisting of: 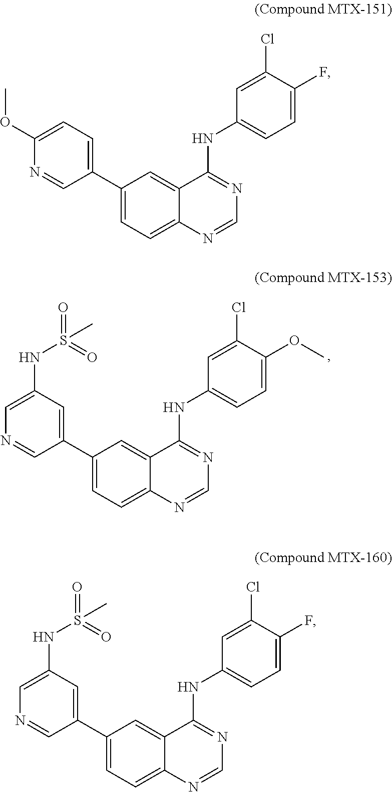 [SMILES: COc1ccc(-c2ccc3ncnc(Nc4ccc(F)c(Cl)c4)c3c2)cn1 ; COc1ccc(Nc2ncnc3ccc(-c4cncc(NS(C)(=O)=O)c4)cc23)cc1Cl ; CS(=O)(=O)Nc1cncc(-c2ccc3ncnc(Nc4ccc(F)c(Cl)c4)c3c2)c1] 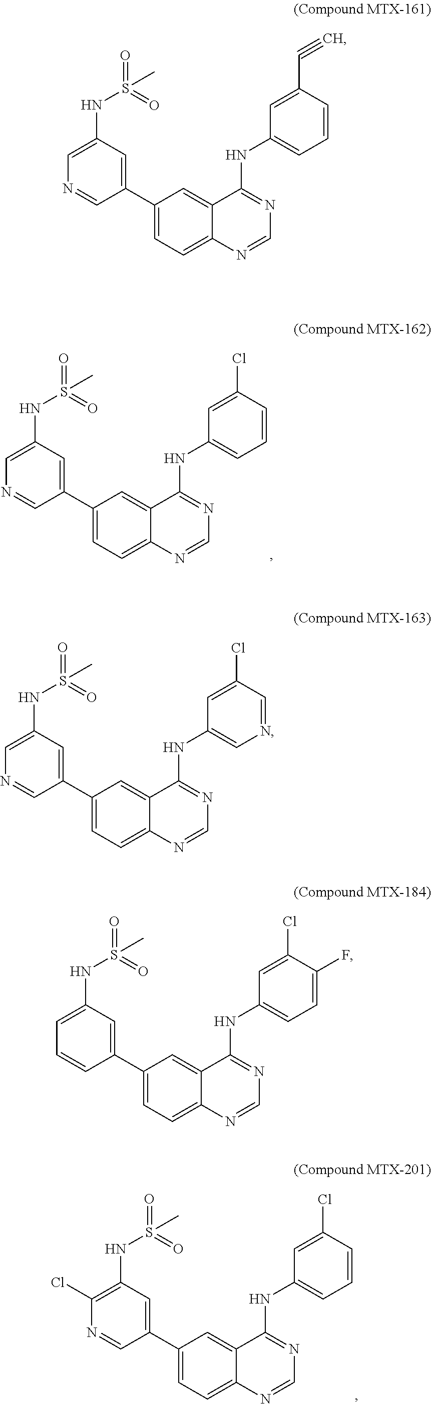 [SMILES: C#Cc1cccc(Nc2ncnc3ccc(-c4cncc(NS(C)(=O)=O)c4)cc23)c1 ; CS(=O)(=O)Nc1cncc(-c2ccc3ncnc(Nc4cccc(Cl)c4)c3c2)c1 ; CS(=O)(=O)Nc1cncc(-c2ccc3ncnc(Nc4cncc(Cl)c4)c3c2)c1 ; CS(=O)(=O)Nc1cccc(-c2ccc3ncnc(Nc4ccc(F)c(Cl)c4)c3c2)c1 ; CS(=O)(=O)Nc1cc(-c2ccc3ncnc(Nc4cccc(Cl)c4)c3c2)cnc1Cl] 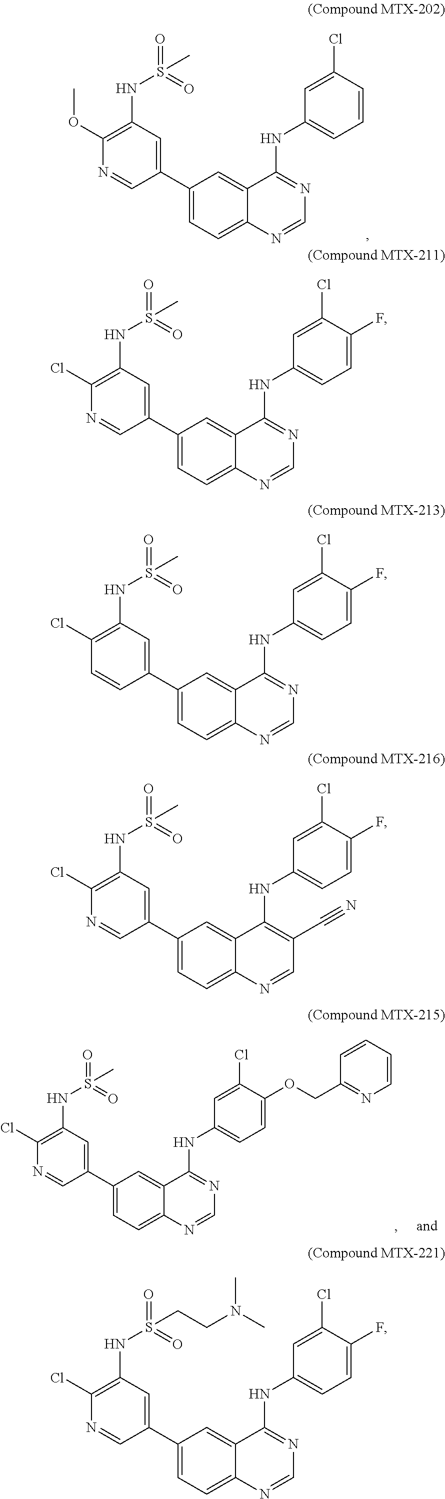 [SMILES: COc1ncc(-c2ccc3ncnc(Nc4cccc(Cl)c4)c3c2)cc1NS(C)(=O)=O ; CS(=O)(=O)Nc1cc(-c2ccc3ncnc(Nc4ccc(F)c(Cl)c4)c3c2)cnc1Cl ; CS(=O)(=O)Nc1cc(-c2ccc3ncnc(Nc4ccc(F)c(Cl)c4)c3c2)ccc1Cl ; CS(=O)(=O)Nc1cc(-c2ccc3ncc(C#N)c(Nc4ccc(F)c(Cl)c4)c3c2)cnc1Cl ; CS(=O)(=O)Nc1cc(-c2ccc3ncnc(Nc4ccc(OCc5ccccn5)c(Cl)c4)c3c2)cnc1Cl ; CN(C)CCS(=O)(=O)Nc1cc(-c2ccc3ncnc(Nc4ccc(F)c(Cl)c4)c3c2)cnc1Cl]
or a pharmaceutically acceptable salt, solvate, or prodrug thereof.

10.-12. (canceled)

13. The method of claim 1, wherein the cancer is associated with mutant BRAF and KRAS.

14. The method of claim 13, wherein said cancer is colorectal cancer, pancreatic, melanoma, or non-small cell lung cancer.

15. The method of claim 1, wherein said patient is a human patient.

16. The method of claim 1, further comprising administering to said patient one or more anticancer agents, or treatments.

17. (canceled)

18. The method of claim 1, wherein the MAPK pathway inhibitor is selected from a BRAF inhibitor, a MEK inhibitor, and an ERK inhibitor.

19. The method of claim 18, wherein the BRAF inhibitor is selected from vemurafenib, LY3009120, Dabrafenib, and LGX818.

20. The method of claim 18, wherein the MEK inhibitor is selected from CH5126766/RO5126766, trametinib, MEK162, and PDO325901.

21. The method of claim 18, wherein the ERK inhibitor is SCH772984.

22. The method of claim 16, wherein said anticancer agent or treatment is a chemotherapeutic agent or radiation therapy.

23. A kit comprising a therapeutically effective combination, the combination comprising a compound of Formula I: 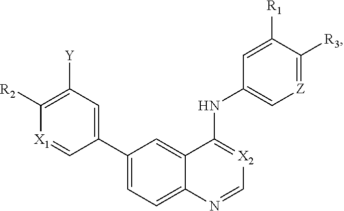
including pharmaceutically acceptable salts, solvates, and/or prodrugs thereof; wherein
R₁ is independently: hydrogen, halogen, or 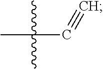 [SMILES: *C#C]
R₂ is independently hydrogen, halogen, or methoxy;
R₃ is independently hydrogen, halogen, methoxy, or pyridin-2-ylmethoxy;
X₁ is independently nitrogen or —CH═;
X₂ is independently nitrogen or —C-nitrile;
Y is independently hydrogen, NHSO₂CH₃, or NHSO₂CH₂CH₂N(CH₃)₂; and
Z is independently nitrogen or —CH═,
at least one MAPK pathway inhibitor;
and instructions for administering said combination to a patient in need thereof having a hyperproliferative disease.

24.-25. (canceled)

26. The kit of claim 23, wherein said cancer is colorectal cancer, pancreatic, melanoma, or non-small cell lung cancer.

27. The kit of claim 23, further comprising one or more anticancer agents.

28. The kit of claim 27, wherein said anticancer agent is a chemotherapeutic agent.

29. (canceled)

30. The kit of claim 23, wherein the MAPK pathway inhibitor is selected from a BRAF inhibitor, a MEK inhibitor, and an ERK inhibitor.

31. The kit of claim 30, wherein the BRAF inhibitor is selected from vemurafenib, LY3009120, Dabrafenib, and LGX818.

32. The kit of claim 30, wherein the MEK inhibitor is selected from CH5126766/RO5126766, trametinib, MEK162, and PDO325901.

33. The kit of claim 30, wherein the ERK inhibitor is SCH772984.

34. The kit of claim 23, wherein the compound of Formula I is a compound selected from the group consisting of: 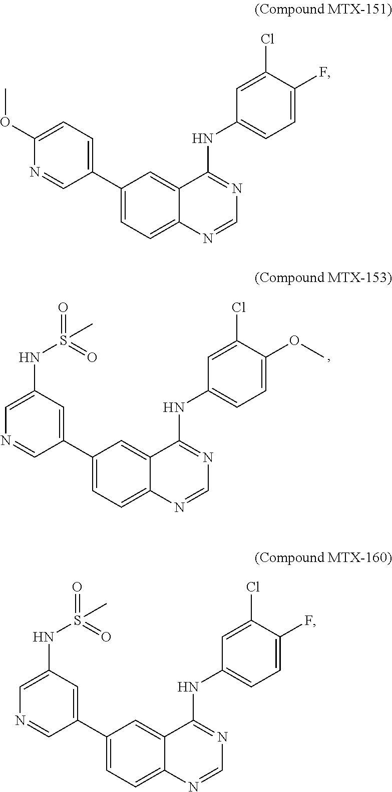 [SMILES: COc1ccc(-c2ccc3ncnc(Nc4ccc(F)c(Cl)c4)c3c2)cn1 ; COc1ccc(Nc2ncnc3ccc(-c4cncc(NS(C)(=O)=O)c4)cc23)cc1Cl ; CS(=O)(=O)Nc1cncc(-c2ccc3ncnc(Nc4ccc(F)c(Cl)c4)c3c2)c1] 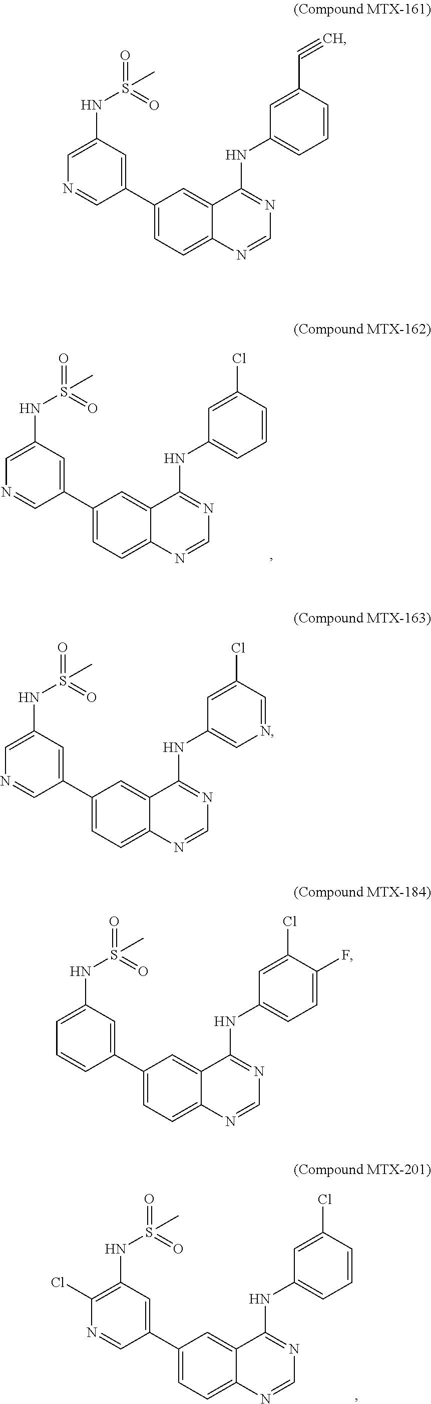 [SMILES: C#Cc1cccc(Nc2ncnc3ccc(-c4cncc(NS(C)(=O)=O)c4)cc23)c1 ; CS(=O)(=O)Nc1cncc(-c2ccc3ncnc(Nc4cccc(Cl)c4)c3c2)c1 ; CS(=O)(=O)Nc1cncc(-c2ccc3ncnc(Nc4cncc(Cl)c4)c3c2)c1 ; CS(=O)(=O)Nc1cccc(-c2ccc3ncnc(Nc4ccc(F)c(Cl)c4)c3c2)c1 ; CS(=O)(=O)Nc1cc(-c2ccc3ncnc(Nc4cccc(Cl)c4)c3c2)cnc1Cl] 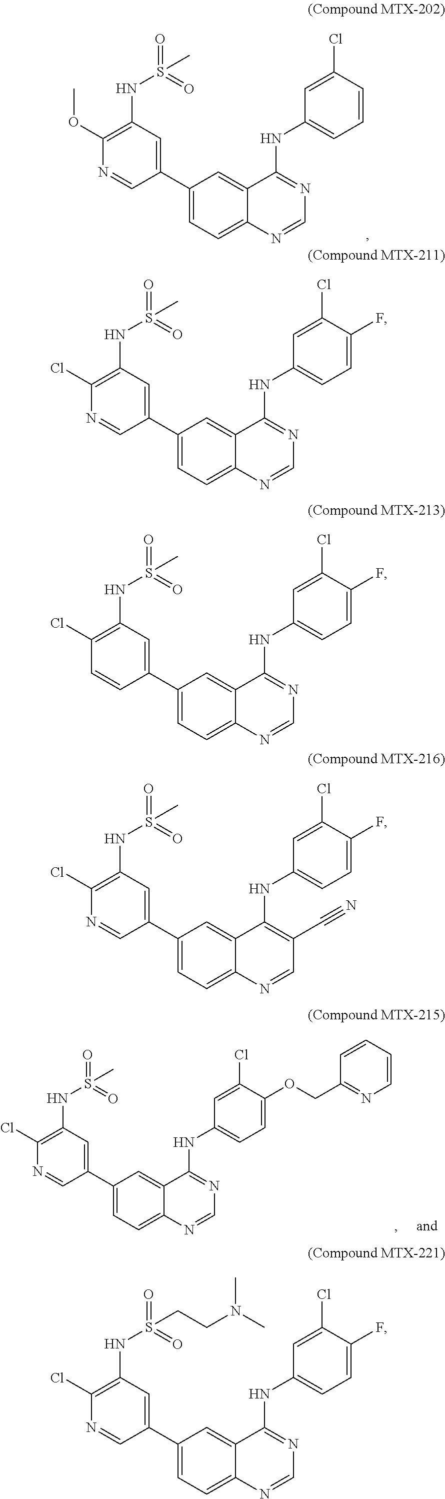 [SMILES: COc1ncc(-c2ccc3ncnc(Nc4cccc(Cl)c4)c3c2)cc1NS(C)(=O)=O ; CS(=O)(=O)Nc1cc(-c2ccc3ncnc(Nc4ccc(F)c(Cl)c4)c3c2)cnc1Cl ; CS(=O)(=O)Nc1cc(-c2ccc3ncnc(Nc4ccc(F)c(Cl)c4)c3c2)ccc1Cl ; CS(=O)(=O)Nc1cc(-c2ccc3ncc(C#N)c(Nc4ccc(F)c(Cl)c4)c3c2)cnc1Cl ; CS(=O)(=O)Nc1cc(-c2ccc3ncnc(Nc4ccc(OCc5ccccn5)c(Cl)c4)c3c2)cnc1Cl ; CN(C)CCS(=O)(=O)Nc1cc(-c2ccc3ncnc(Nc4ccc(F)c(Cl)c4)c3c2)cnc1Cl]
or a pharmaceutically acceptable salt, solvate, or prodrug thereof.

35. A combination for the treatment of a cancer, the combination comprising:
a compound of Formula I: 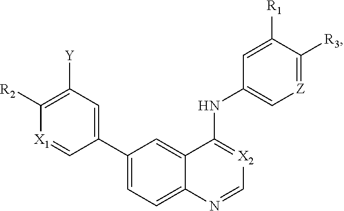
including pharmaceutically acceptable salts, solvates, and/or prodrugs thereof; wherein
R₁ is independently: hydrogen, halogen, or 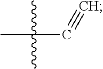 [SMILES: *C#C]
R₂ is independently hydrogen, halogen, or methoxy;
R₃ is independently hydrogen, halogen, methoxy, or pyridin-2-ylmethoxy;
X₁ is independently nitrogen or —CH═;
X₂ is independently nitrogen or —C-nitrile;
Y is independently hydrogen, NHSO₂CH₃, or NHSO₂CH₂CH₂N(CH₃)₂; and
Z is independently nitrogen or —CH═;
and at least one MAPK pathway inhibitor.

36. The combination of claim 35, wherein the compound of Formula I is selected from the group consisting of: 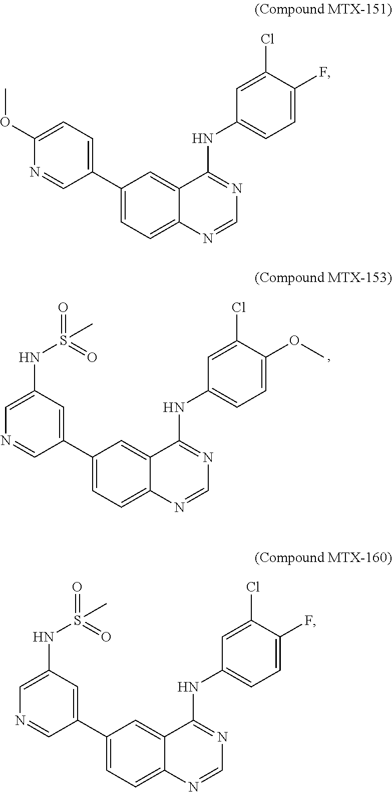 [SMILES: COc1ccc(-c2ccc3ncnc(Nc4ccc(F)c(Cl)c4)c3c2)cn1 ; COc1ccc(Nc2ncnc3ccc(-c4cncc(NS(C)(=O)=O)c4)cc23)cc1Cl ; CS(=O)(=O)Nc1cncc(-c2ccc3ncnc(Nc4ccc(F)c(Cl)c4)c3c2)c1] 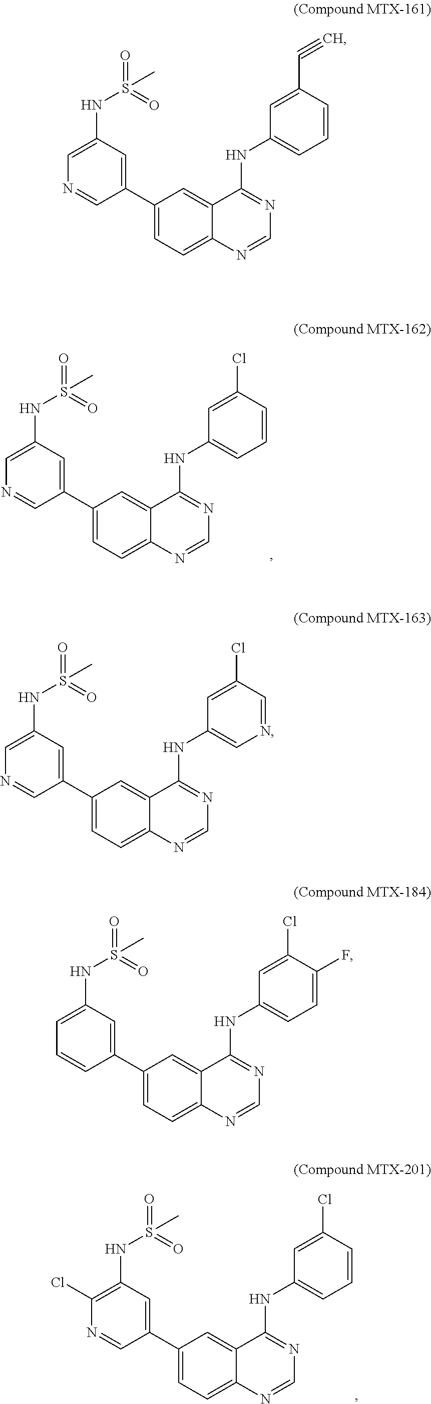 [SMILES: C#Cc1cccc(Nc2ncnc3ccc(-c4cncc(NS(C)(=O)=O)c4)cc23)c1 ; CS(=O)(=O)Nc1cncc(-c2ccc3ncnc(Nc4cccc(Cl)c4)c3c2)c1 ; CS(=O)(=O)Nc1cncc(-c2ccc3ncnc(Nc4cncc(Cl)c4)c3c2)c1 ; CS(=O)(=O)Nc1cccc(-c2ccc3ncnc(Nc4ccc(F)c(Cl)c4)c3c2)c1 ; CS(=O)(=O)Nc1cc(-c2ccc3ncnc(Nc4cccc(Cl)c4)c3c2)cnc1Cl] 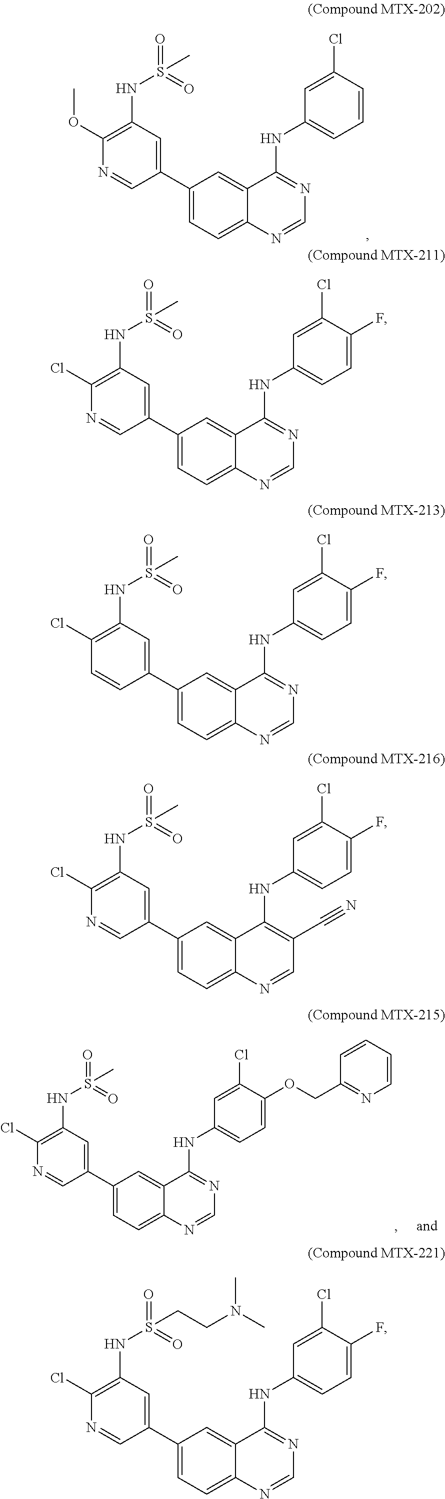 [SMILES: COc1ncc(-c2ccc3ncnc(Nc4cccc(Cl)c4)c3c2)cc1NS(C)(=O)=O ; CS(=O)(=O)Nc1cc(-c2ccc3ncnc(Nc4ccc(F)c(Cl)c4)c3c2)cnc1Cl ; CS(=O)(=O)Nc1cc(-c2ccc3ncnc(Nc4ccc(F)c(Cl)c4)c3c2)ccc1Cl ; CS(=O)(=O)Nc1cc(-c2ccc3ncc(C#N)c(Nc4ccc(F)c(Cl)c4)c3c2)cnc1Cl ; CS(=O)(=O)Nc1cc(-c2ccc3ncnc(Nc4ccc(OCc5ccccn5)c(Cl)c4)c3c2)cnc1Cl ; CN(C)CCS(=O)(=O)Nc1cc(-c2ccc3ncnc(Nc4ccc(F)c(Cl)c4)c3c2)cnc1Cl]
or a pharmaceutically acceptable salt, solvate, or prodrug thereof.

37. The combination of claim 35, wherein the MAPK pathway inhibitor is selected from a BRAF inhibitor, a MEK inhibitor, and an ERK inhibitor.

38. The combination of claim 37, wherein the BRAF inhibitor is selected from vemurafenib, LY3009120, Dabrafenib, and LGX818.

39. The combination of claim 37, wherein the MEK inhibitor is selected from CH5126766/RO5126766, trametinib, MEK162, and PDO325901.

40. The combination of claim 37, wherein the ERK inhibitor is SCH772984.
